(19) United States
(12) Patent Application Publication
(10) Pub. No.: US 2023/0225586 A1
(43) Pub. Date: Jul. 20, 2023

(54) HEMOSTASIS METHODS AND APPARATUSES

(71) Applicant: PROCEPT BioRobotics Corporation, Redwood City, CA (US)

(72) Inventors: Surag MANTRI, East Palo Alto, CA (US); Kevin Patrick STAID, Lowell, MA (US); James Luis BADIA, Redwood City, CA (US); William TOLMASOFF, Oakland, CA (US); Friedrich HO, Mountain View, CA (US)

(73) Assignee: PROCEPT BioRobotics Corporation, Redwood City, CA (US)

(21) Appl. No.: 18/001,570

(22) PCT Filed: Jul. 8, 2021
(86) PCT No.: PCT/US2021/040943

§ 371 Date: Dec. 12, 2022

Related U.S. Application Data
(60) Provisional application No. 63/049,523, filed on Jul. 8, 2020.

(51) Int. Cl. A61B 1/00; A61B 1/06; A61B 1/015; A61B 18/24; A61B 1/07

(52) U.S. Cl. CPC A61B 1/00082; A61B 1/0638; A61B 1/063; A61B 1/015; A61B 18/24; A61B 1/07; A61B 2018/00589

(57) ABSTRACT

A probe is configured with a flushing port and an evacuation port to establish a flow path to remove blood from a resected tissue. The probe comprises a balloon configured to expand and contact the resected tissue to compress filaments and improve access to the underlying blood vessels for coagulation with an energy source. An endoscope can be used to view the tissue, and the balloon may comprise a transparent material or a viewing port to allow imaging of the bleeding tissue through the balloon. The probe may have a light source to illuminate the tissue with a beam oriented at an oblique angle to the tissue surface, which can decrease interference from blood and may allow more localized coagulation of the blood vessel.

### RELATED APPLICATIONS

This application is a 371 national phase of PCT/US2021/040943, filed Jul. 8, 2021, published as WO 2022/011177 on Jan. 13, 2022, and claims the benefit under 35 U.S.C. § 119(e) of U.S. Provisional Patent Application No. 63/049,523, filed Jul. 8, 2020, and titled “HEMOSTASIS METHODS AND APPARATUSES,” which is incorporated, in its entirety, by this reference.

### BACKGROUND

Work in relation to the present disclosure suggests that prior approaches to treating bleeding tissue with energy can be less than ideal in at least some respects. In some instances, bleeding tissue may comprise tissue with rough surfaces, which can make the bleeding tissue somewhat more difficult to treat. For example, blood may coagulate on the rough surface and decrease visibility of the underlying surface. Also, blood can potentially obscure light from an energy source such as a laser, which can result in less than ideal delivery of the light energy to a target tissue, such as a ruptured blood vessel. The rough surface may also make the distribution of light energy provided to the tissue somewhat less evenly distributed and the resulting coagulation less uniform than would be ideal.

Water jets can be used resect tissue with decreased bleeding. For example, a water jet can selectively resect tissue such as a glandular prostate tissue while leaving collagenous tissue such as blood vessels substantially intact. However, in some instances tissue resection with a water jet can lead to the penetration of blood vessels which can result in bleeding. In some instances, soft tissue such as glandular tissue also has collagenous connective tissue fibers that support the soft tissue. Work in relation to the present disclosure suggests that the resection of soft tissue with a water jet can leave collagenous tissue fibers after the soft tissue such as glandular tissue has been removed. These remaining collagenous tissue fibers can collect blood and interfere with hemostasis treatment in at least some instances. For example, the collagenous fibers can decrease visibility of a blood vessel, which may make placement of a hemostasis treatment less accurate than would be ideal. Also, blood collected by the fibers can at least partially interfere with the delivery of laser energy to the underlying blood vessel in at least some instances.

In light of the above, improve methods and apparatus are needed that ameliorate at least some of the limitations of the prior approaches.

### SUMMARY

The presently disclosed, probes, methods and apparatuses can provide improved hemostasis to bleeding tissue and can be used for the treatment of bleeding tissue with residual collagenous fibers. In some embodiments, a probe is configured with a flushing port and an evacuation port configured to establish a flow path to remove blood from a resected tissue. In some embodiments, the probe comprises a balloon configured to expand and contact the resected tissue to compress filaments and improve access to the underlying blood vessels for coagulation with an energy source such as a laser beam. An endoscope can be used to view the tissue, and the balloon may comprise a transparent material to allow imaging of the bleeding tissue through the balloon. The endoscope may comprise a viewing port within the balloon or external to the balloon in order to image the tissue through the balloon. In some embodiments, the probe comprises a light source configured to illuminate the tissue with a beam oriented at an oblique angle to the tissue surface, which can decrease interference from blood and may allow more localized coagulation of the blood vessel. The probe can be manipulated in many ways and can be connected to one or more of a handpiece or a robotic linkage to move the energy source.

In some embodiments, the probe is coupled to a robotic linkage configured to receive instructions from a processor. The processor can be configured to receive an input corresponding to a location of a ruptured blood vessel and to scan the energy source with a pattern in relation to the location. The input can be determined in many ways and may comprise one or more of an input from an ultrasound image, a Doppler ultrasound image, an endoscopic image, an aiming beam on a probe, or a user input on an image of the tissue. In some embodiments, the processor is configured to scan the energy source at a distance from the location, which can be helpful in coagulating underlying blood at a distance from the ruptured opening to the blood vessel.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows an apparatus suitable for performing a prostatic tissue hemostasis procedure in accordance with embodiments;

FIGS. 2A to 2D illustrate use of the apparatus of FIG. 1 in performing prostatic tissue hemostasis;

FIG. 2E shows a treatment probe with an endoscope configured to view a treatment site from an interior of a balloon 24, in accordance with some embodiments;

FIG. 2F shows a resection profile along a prostate and a resected blood vessel, in accordance with some embodiments;

FIG. 2G shows a scan pattern suitable for treating a blood vessel away from the opening along the resection profile, in accordance with some embodiments;

FIG. 2H shows a spiral energy scan pattern away from a vessel opening, in accordance with some embodiments;

FIGS. 3A and 3B show a system to treat a patient in accordance with embodiments;

FIG. 4A shows blood flow and a Doppler ultrasound probe, in accordance with some embodiments;

FIG. 4B shows a Doppler ultrasound image on a display for a probe as in FIG. 4A, in accordance with some embodiments;

FIG. 5 shows an aiming laser beam on a laser energy delivery probe, in accordance with some embodiments;

FIG. 6 shows an inflated balloon with a substantially non-attenuating fluid between a laser energy treatment probe and a target site, in accordance with embodiments;

FIG. 7A shows an inflated balloon placed against a tissue resection profile, in accordance with some embodiments;

FIG. 7B shows fluid flow over a tissue resection profile and plume of blood, in accordance with some embodiments;

FIG. 8A shows extendable optical fibers deployed from an end of a lumen, in accordance with some embodiments;

FIG. 8B shows a tissue engagement structure comprising a roller coupled to an optical fiber, in accordance with some embodiments;

FIG. 9A shows a side emitting laser energy delivery probe, in accordance with some embodiments;

FIG. 9B shows a probe comprising an array of optical fibers to deliver laser energy to tissue, in accordance with some embodiments;

FIG. 10 shows an optical fiber coupled to a conical mirror, in accordance with some embodiments;

FIG. 11 shows a double balloon comprising an inner balloon and an outer balloon configured to define a fluid flow channel between the inner balloon and the outer balloon, in accordance with some embodiments;

FIG. 12A shows a combination treatment probe comprising an optical fiber to release energy to the tissue at a first location on the probe and a nozzle to release a water jet at a second location on the probe, in accordance with some embodiments;

FIG. 12B shows a treatment probe comprising a nozzle coupled to a pressurized jet lumen deliver a water jet to tissue and an optical fiber outside the lumen, in accordance with some embodiments;

FIG. 12C shows a high-pressure lumen coupled to a nozzle to release a water jet and an optical fiber to treat tissue with light energy, in which the optical fiber is located in the high-pressure lumen, in accordance with some embodiments;

FIG. 12D shows a probe comprising a light source and a nozzle at different locations on a probe, in which the light source and nozzle are oriented so as to at least partially overlap at a tissue location;

FIG. 12E shows a probe comprising a light source and a nozzle in which the light source and the nozzle are space apart axially;

FIG. 13 shows an oblique angle of incidence of laser energy with respect to a tissue resection profile to delivery laser energy to a blood vessel beneath the tissue resection profile, in accordance with some embodiments; and

FIG. 14 shows a balloon extending around a light energy delivery port of an optical fiber delivery probe, in accordance with some embodiments;

FIG. 15 shows a probe comprising an electrode, in accordance with some embodiments;

FIG. 16 shows a probe comprising an optical fiber coupled to an aiming laser and a treatment laser;

FIG. 17 shows tissue resection zones in accordance with some embodiments;

FIG. 18 shows a method of reducing bleeding, in accordance with some embodiments;

FIG. 19 shows a probe and selective tissue resection zones, in accordance with some embodiments;

FIG. 20 shows resection of blood vessels and water jet intensities corresponding to a collagen removal zone, a collagen disruption zone and a collagen preservation zone, in accordance with some embodiments;

FIG. 21 shows an endoscopic image of a resected human prostate, in accordance with some embodiments;

FIG. 22 shows a wire loop extending from a cannula, in accordance with some embodiments;

FIG. 23 shows a wire loop having an adjustable loop diameter, in accordance with some embodiments;

FIG. 24 shows a wire loop having an adjustable loop diameter with electrodes, in accordance with some embodiments;

FIG. 25 shows a hollow wire loop having an optical fiber extending therethrough, in accordance with some embodiments;

FIG. 26A shows an adjustable active electrode loop and a snare extending from a probe, in accordance with some embodiments;

FIG. 26B shows an adjustable active electrode in an expanded configuration and a snare extending from a probe, in accordance with some embodiments;

FIG. 27A shows a probe with a snare in a first configuration, in accordance with some embodiments;

FIG. 27B shows a robe with a snare in a second configuration, in accordance with some embodiments;

FIG. 28 shows a resectoscope sheath usable with a probe, in accordance with some embodiments;

FIG. 29A shows a probe and an active electrode rotated in a first configuration, in accordance with some embodiments;

FIG. 29B shows a probe and an active electrode rotated in a second configuration, in accordance with some embodiments;

FIG. 30 shows a probe with an active helical adjustable loop, in accordance with some embodiments;

FIG. 31A shows a probe with an active adjustable loop and snare in accordance with some embodiments;

FIG. 31B shows a probe with an active adjustable loop in an expanded configuration, in accordance with some embodiments;

FIG. 32 shows a probe with an active electrode adjustable loop, in accordance with some embodiment;

FIG. 33A shows a dog prostate tissue section post aqua ablation with about six weeks of healing, in accordance with some embodiments;

FIG. 33B shows a dog prostate tissue section post aqua ablation with six weeks of healing, in accordance with some embodiments;

FIG. 33C shows a dog prostate tissue section post aqua ablation with six weeks of healing, in accordance with some embodiments;

FIG. 33D shows a dog prostate tissue section post aqua ablation with six weeks of healing, in accordance with some embodiments;

FIG. 33E shows a histological tissue section 3300 of a collagenous tissue bundle of fibers, in accordance with some embodiments;

FIG. 34 shows geometric control of an energy loop, in accordance with some embodiments; and

FIG. 35 shows cross sectional geometries that may be configured to target anatomy with a structure such as a loop, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The presently disclosed methods and apparatus are well suited for treating bleeding tissue that has been treated with an energy source. The energy source may comprise one or more of a laser beam, a water jet, an electrode, ultrasound, high intensity focused ultrasound, mechanical vibrations, radiofrequency (RF) energy an ultrasound transducer, microwave energy, cavitating energy such as a cavitating water jet or ultrasonic cavitations.

Work in relation to the present disclosure suggests that the resection of tissue with a water jet can provide elongate collagenous filaments on the resected surface that are somewhat more resistant to tissue resection than other types of tissue. The presently disclosed methods and apparatus are well suited for treating tissue under such filaments, for example to provide tissue coagulation and hemostasis. For example, with the resection of prostate tissue for the treatment of benign prostate hyperplasia (“BPH”), the water jet resection can selectively resect tissue such as glandular tissue, while leaving collagenous tissue filaments. These collagenous filaments may also be referred to herein as fluffies because of the “fluffy” appearance of the collagenous fibers.

While embodiments of the present disclosure are specifically directed to treatment of the prostate, certain aspects of the disclosure may also be used to treat and modify other organs and tissue such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone, as well as body lumens and passages such as the sinuses, ureter, colon, esophagus, lung passages, blood vessels, and throat. The devices disclosed herein may be inserted through an existing body lumen, or inserted through an opening created in body tissue.

The presently disclosed methods and apparatuses can be configured in many ways to promote at least partial closure of blood vessels to decrease bleeding. In some embodiments, bleeding tissue is infused with laser absorption accelerator, such as a wavelength specific chromophore, which can be provided through one or more lumens used to provide fluid to a treatment location. The presently disclosed methods and apparatus can be configured to use decreased amounts of energy to promote hemostasis, for example with less energy than could be used for tissue ablation.

The light energy can be provided by any suitable light source and may comprise any suitable wavelength or combination of wavelengths. For example, the light energy may comprise one or more of ultraviolet, visible, infrared, or near infrared electromagnetic energy. The light source may comprise one or more of a laser, a laser diode, a super luminescent diode, light bulb, a flash bulb, or a halogen bulb, for example. While the light source can be configured in many ways, in some embodiments, the light source is coupled to one or more optical fibers, and the light energy released from the one or more optical fibers toward the tissue.

In some embodiments, a probe is configured to emit light having a wavelength for decreased tissue penetration for hemostasis, which can decrease nerve damage around delicate tissue structures such as the capsule of the prostate.

The treatment probe can be configured in many ways, and in some embodiments is configured to release a fluid with relatively low laser attenuation compared to the target tissue. The fluid may comprise one or more of gas or a liquid. The gas may comprise air or carbon dioxide (CO₂), for example. The liquid may comprise water, saline, or a mixture of water with another material.

Work in relation to the present disclosure suggests that the prostate may tend to bleed more generally anteriorly toward the prostate capsule, for example at locations corresponding to 10 o'clock and 2 o'clock with the patient on a support. In some embodiments the target tissue is insufflated with an optically transmissive fluid and the treatment energy such as laser energy is directed to regions corresponding to 10 o'clock and 2 o'clock, for example with a treatment extending approximately ±20° at each location.

In some embodiments, a dye comprising a chromophore is delivered to the tissue. In some embodiments, the dye is delivered to the tissue with a fluid comprising of one or more of the water jet, a flushing fluid, or an insufflation fluid tissue. The dye may comprise a chromophore with a peak absorbance near a wavelength of the light energy (e.g. a treatment wavelength corresponding to an absorbance of at least half of the peak absorbance of the chromophore). This may provide depth of penetration control and surface temperatures to promote one or more of clot formation or vessel sealing, without excessive depth of penetration so as to decrease damage to tissues near the treatment site.

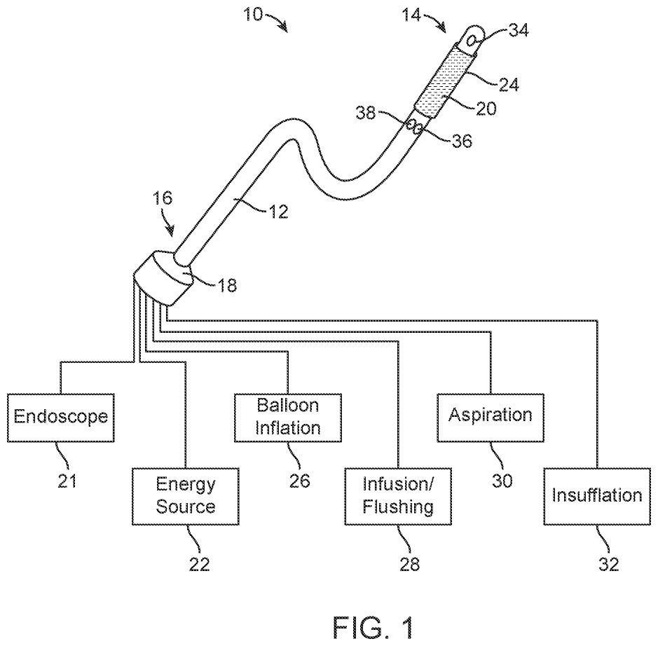

Referring to FIG. 1, an exemplary prostatic tissue hemostasis apparatus 10 constructed in accordance with the principles of the present disclosure comprises a catheter assembly generally including a shaft 12 having a distal end 14 and a proximal end 16. In some embodiments, the shaft 12 comprises one a polymeric extrusion or metallic tubes (and combinations thereof) including one, two, three, four, or more axial lumens extending from a hub 18 at the proximal end 16 to locations near the distal end 14. The shaft 12 may be stiff, flexible or rigid. In some embodiments, the shaft 12 has a length in the range from 15 cm to 25 cm and a diameter in the range from 1 mm to 15 mm, usually from 2 mm to 10 mm. The shaft will typically have sufficient column strength so that it may be introduced upwardly through the male urethra, as described in more detail below.

In some embodiments, the shaft includes an energy source positioned in the energy delivery region 20, where the energy source can be any one of a number of specific components as discussed in more detail below. In some embodiments an inflatable balloon 24 is positioned near the distal end 14 of the shaft and extend over an energy release element at energy delivery region 20. The balloon is connected through one of the axial lumens to a balloon inflation source 26, the balloon inflation source can be of a piston driven fluid displacement device such as a fluid pump (e.g., peristaltic, gear, vane, piston, including a manual or motorized syringe delivering liquid or gas to a preselected volume or pressure to the balloon), connected through the hub 18. In addition to the energy source 22 and the balloon inflation source 26, the hub optionally further includes connections for an infusion/flushing source 28, an aspiration (a vacuum) source 30, and/or an insufflation (pressurized gas such as CO₂ or a liquid such as saline) source 32. In the exemplary embodiment, the infusion or flushing source 28 can be connected through an axial lumen (not shown) to one or more delivery ports 34 proximal or distal to the balloon 24 and distal to the energy delivery region 20. The aspiration source 30 can be connected to a second port or opening 36, which can be positioned proximally of the energy delivery region 20 or distally to the energy delivery region 20. The insufflation source 32 can be connected to an additional port 38, which can be located proximal or distally relative to the energy delivery region. It will be appreciated that the locations of the ports 34, 36, and 38 are not critical, although certain positions may result in particular advantages described herein, and that the lumens and delivery means could be provided by additional catheters, tubes, and the like, for example including coaxial sleeves, sheathes, and the like which could be positioned over the shaft 12.

In some embodiments, the hemostasis apparatus 10 comprise an endoscope 21 to allow visualization of the tissue proximate energy delivery region 20. The endoscope 21 is configured to allow visualization of the treatment site. The endoscope 21 may comprise an optical port for viewing the treatment site. The optical port may comprise one or more lenses to receive light from the treatment site, for example. The optical port may be located within balloon 24 or external to balloon 24. The balloon 24 comprises an optically transmissive material to allow visualization of the treatment site through the balloon with at least one wavelength of light. In embodiments where the viewing port is located within the balloon 24, the endoscope is configured to view the treatment site from an interior of the balloon. In embodiments where the endoscope viewing port is located outside the balloon, the endoscope views the treatment site with light transmitted through the balloon at two or more locations, in which one location is near the treatments site and the other location is near the endoscope.

The balloon on the probe can be configured in many ways. In some embodiments, the balloon in the narrow profile configuration comprises one or more of an approximately cylindrical shape within 25% of the probe diameter, a balloon comprising a diameter larger than a shaft of the probe and a tapered shape profile near a distal end of the balloon to facilitate advancement of the probe, or a balloon wrapped around the shaft to decrease a cross-sectional size of the balloon.

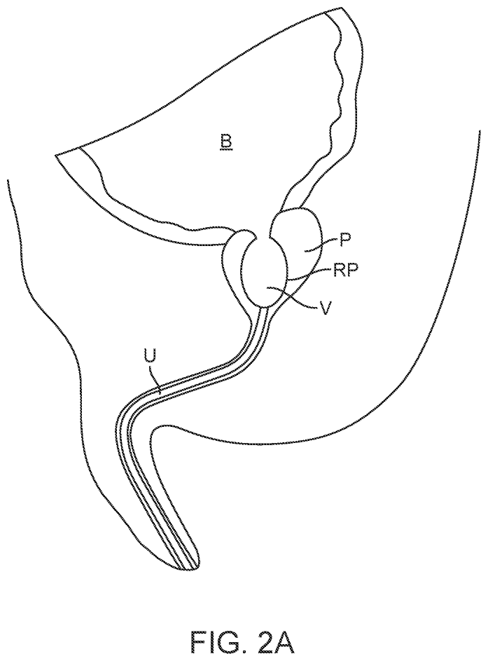

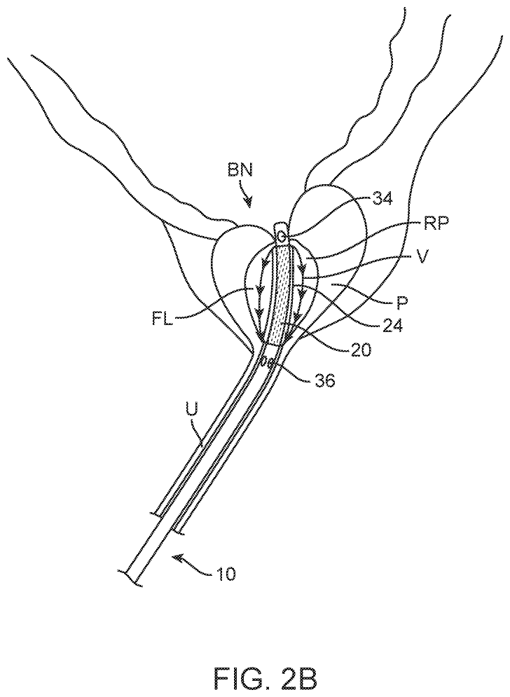

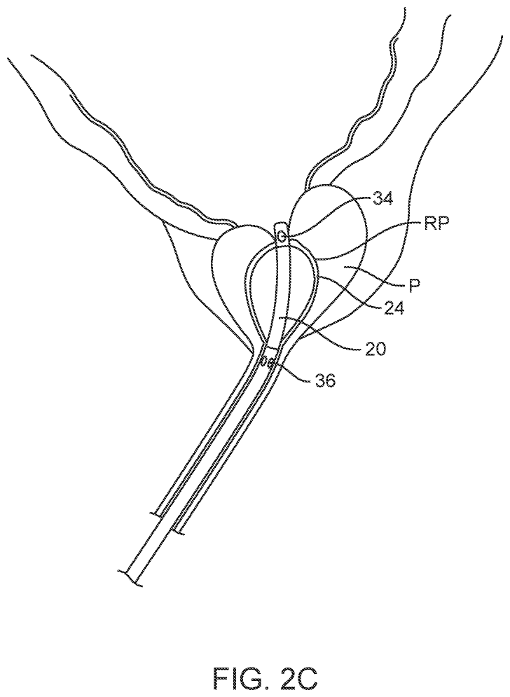

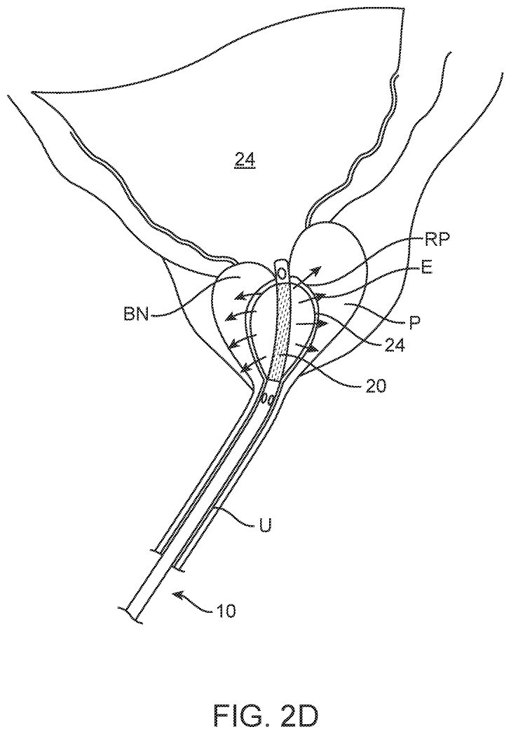

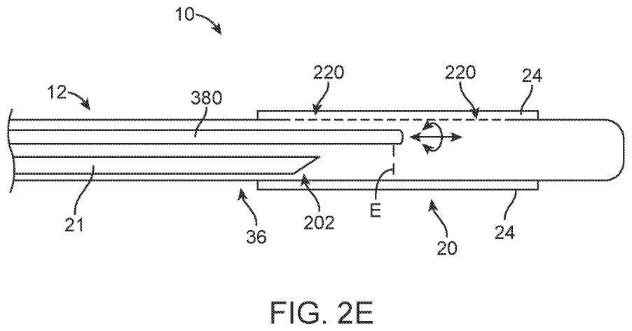

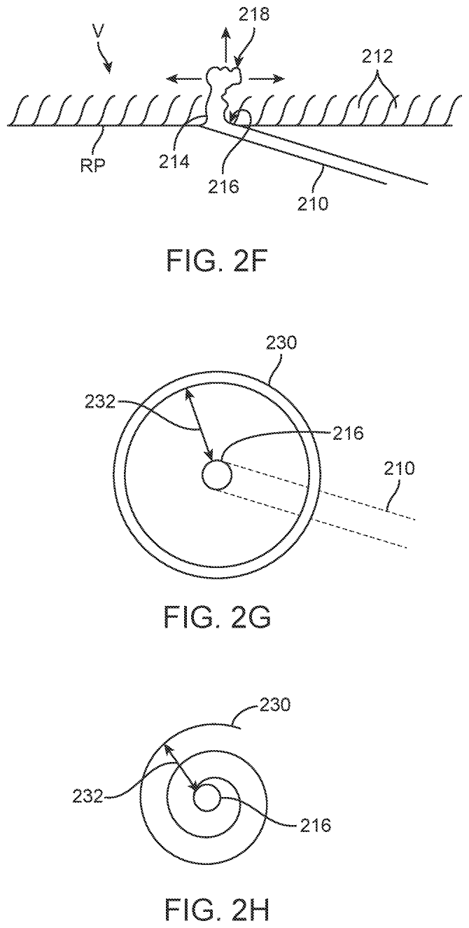

Referring now to FIGS. 2A to 2D, the prostatic tissue hemostasis apparatus 10 is introduced through the male urethra U to a region within the prostate P which is located immediately distal to the bladder B. The anatomy is shown in FIG. 2A. In some embodiments, a volume V of the tissue of the prostate P has been resected to a resection profile RP. While the prostate can be resected in many ways, in some embodiments the prostatic tissue is resected with a water jet.

As shown in FIG. 2B, once the catheter 10 has been positioned so that the balloon is placed in proximity to the resection profile RP, fluid flow FL can be provided to the resected prostate. In some embodiments, the fluid flow comprises the release of fluid through port 34 on one side of the balloon, e.g. distal to the balloon 24, and evacuation of the fluid on another side of the balloon, e.g. proximal to the balloon, although the arrangement can be reversed. This flow of fluid can facilitate the removal of blood that may otherwise interfere with treatment of the blood vessels with energy and allow improved visualization of the source of bleeding.

As shown in FIG. 2C, the balloon 24 is inflated. The inflation of the balloon can displace material such as blood from the resected volume of tissue V. In some embodiments, the position of the prostatic tissue hemostasis apparatus 10 is fixed and stabilized within the resected volume V so that the energy delivery region 20 is positioned within the prostate P.

As shown in FIG. 2D, after the balloon 24 has been inflated, energy E can be delivered into the prostate for hemostasis, as shown by the arrows in FIG. 2D. Once the energy has been delivered for a time and over a desired surface region, the energy region can be stopped and the prostate is treated for hemostasis to substantially decrease bleeding. In embodiments in which the source of the energy is light based, it may also be configured to use alternating signals to apply treatment and diagnose the efficacy of that treatment. For example, if laser energy is applied, it can use alternating wavelength signals of energy for treatment and transmission/receiving signals to measure the temperature of the zone of hemostasis. Further embodiments may include two or more fibers of different wavelengths, such that the first fiber may be used to apply treatment while the second fiber may be used to measure the temperature of the zone of hemostasis simultaneously by infrared, tissue color change, visual cues or other means. A positive feedback loop may be created such that the first fiber will halt treatment once the tissue temperature has reached the target required to achieve hemostasis.

FIG. 2E shows a treatment probe with an endoscope configured to view a treatment site from an interior of a balloon 24. The endoscope 21 comprises a viewing port 202 oriented to receive light from the treatment region and image the treatment region with the balloon 24 inflated. The port 36 is located on one side of the balloon, e.g. proximally. The energy deliver region 20 comprises carrier 380 configured to deliver energy E along an energy delivery path as described herein. The carrier 380 can be configured in many ways to deliver energy and can be configured to translate along an elongate axis of the carrier and to rotate about the elongate axis of the carrier 380, for example. The carrier 380 can be coupled to a linkage as described herein, or operated manually, for example with a handle. A wall defining one or more lumens of the shaft 12 may comprise an aperture to inflate the balloon and allow the treatment site to be imaged with the viewing port of the endoscope. Alternatively or in combination, openings 220 can be provided to allow inflation of the balloon 24.

Although FIG. 2E shows the viewing port 202 of the endoscope 21 located on an interior of the balloon 24, in some embodiments the endoscope viewing port is located on an exterior of the balloon. For example, the endoscope viewing port can be located proximally to the balloon and the port 36 located proximal to the viewing port to establish a fluid flow. The endoscope 21 and balloon 24 can be configured to view tissue through the balloon, and the balloon and inflation fluid may comprise a substantially transparent material, such as a substantially transparent liquid or a gas. The energy source and carrier 380 can be rotated and translated within the balloon to deliver energy to the tissue with the endoscope viewing port oriented toward the tissue so as to view the tissue through a first portion of the balloon proximate the endoscope viewing port and through a second portion of the balloon proximate the tissue. The fluid flow between the flush port and evacuation port can be established as described herein, so as to evacuate material such as blood and clots from the surgical site to improve visualization and access to tissue near the blood vessel and in some embodiments the blood vessel itself.

FIG. 2F shows a resection profile RP along a prostate and a resected blood vessel 210. In some embodiments, the profile along the remaining tissue of the resection profile RP comprises tissue filaments 212 (“fluffies”). In some embodiments, the energy source used to selectively resect tissue while leaving other types of tissue. Work in relation to the present disclosure suggests that tissue resection with a water jet may remove some types of tissue, e.g. glandular tissue, more quickly than other tissue such as supportive tissue comprising relatively greater amounts of collagen fibers. The blood vessel extends to an opening 216 in the blood vessel generally along the resection profile. The opening of the blood vessel can release blood 214 into the volume V of resected tissue. The filaments can collect blood 214 and may comprise molecular structures that tend to induce blood clotting. In some embodiments, a plume 218 of blood is released into the resected volume V. The blood vessel is shown beneath the resection profile inclined at an oblique angle relative to the resected tissue profile. Because the blood released into the blood vessel can collect near the opening, work in relation to the present disclosure suggests that it can be helpful to treat tissue away from the opening to the blood vessel, so as to decrease energy absorbed by blood released from the vessel into the filaments and the resection volume.

FIG. 2G shows an energy scan pattern 230 suitable for treating a blood vessel 210 away from the opening along the resection profile. The distance 232 can be any suitable distance, for example a distance within a range from about 1 mm to about 10 mm, for example. The scan pattern may comprise one or more shapes, such as an annular scan pattern, a plurality of annuli, an ellipsoidal pattern, a spiral scan pattern or a raster scan pattern, and combinations thereof. A plurality of scan patterns can be applied to the tissue, for example a plurality of annuli of decreasing diameter.

In some embodiments, the scan pattern may result in coagulation of the blood within the tissue and the blood vessel at a depth into the resected tissue and the vessel. Coagulating the blood deeper into the tissue, such as greater than 3 mm, 5 mm, 8 mm, 10 mm, or 15 mm into the tissue or vessel, aids in coagulation and stopping the bleeding out of the opening in the vessel as compared to only surface treatment.

FIG. 2H shows a spiral energy scan pattern 230 away from a vessel opening 216. The scan pattern 230 comprises a portion located a distance from the vessel opening 216 to decrease interference from blood released through the opening to the blood vessel.

The scan patterns 230 shown in FIGS. 2G and 2H can be implemented in many ways. For example, a physician can direct a low power visible light energy beam to target the vessel opening, e.g. by using the energy delivery element as a laser pointer. When the low power visible light energy beam is aligned with the vessel opening, the user can operate an input such as a foot pedal. In response to the user input that the aiming beam is aligned with the target opening, the processor can direct the energy delivery element to deliver the scan pattern 230 around the opening to the vessel.

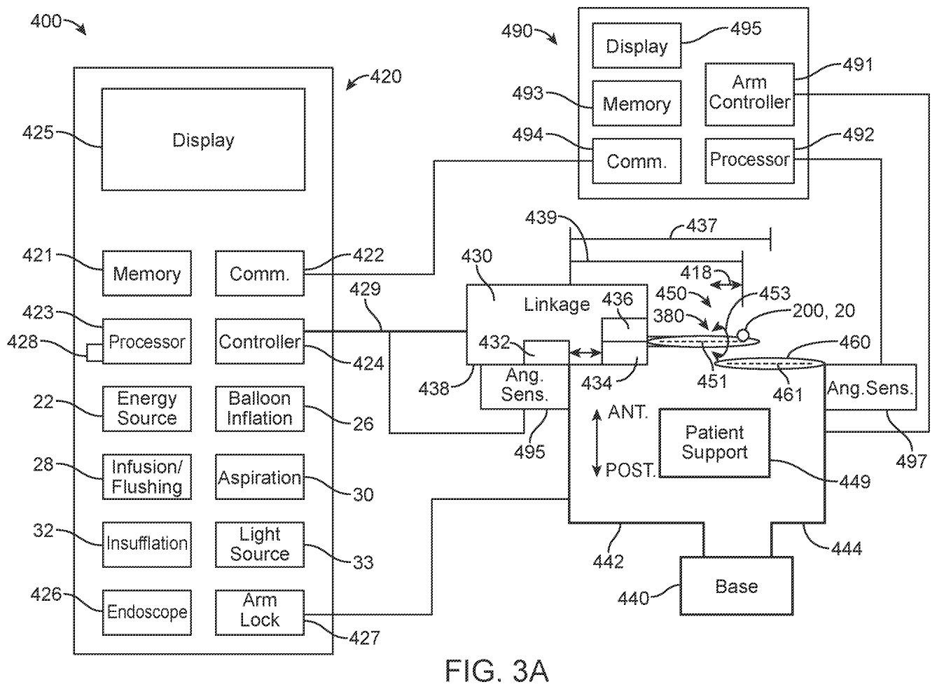

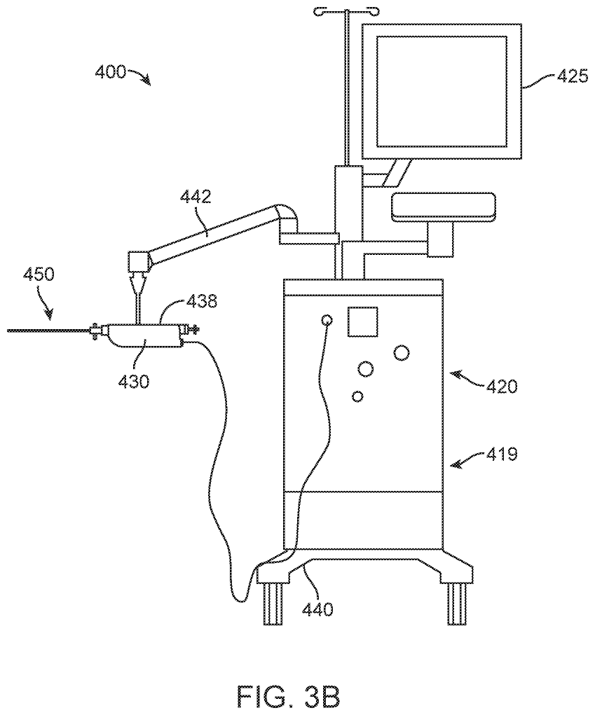

FIGS. 3A and 3B show a system to treat a patient in accordance with embodiments. The system 400 comprises a treatment probe 450 and may optionally comprise an imaging probe 460. The treatment probe 450 is coupled to a console 420 and a linkage 430. The imaging probe 460 is coupled to an imaging console 490. The patient treatment probe 450 and the imaging probe 460 can be coupled to a common base 440. The patient is supported with the patient support 449. The treatment probe 450 is coupled to the base 440 with an arm 442. The imaging probe 460 is coupled to the base 440 with an arm 444. The arm 444 may comprise a robotic arm, such as a 5 to 8 degree of freedom robotic arm, for example. Examples of robotic arms suitable for incorporation with the present disclosure include robotic arms commercially available from Intuitive Surgical, e.g. the DaVinci system, robotic arms commercially available from Auris Health, e.g. the Monarch Surgical Robot, robotic arms for industrial and medical applications, such as robotic arms commercially available from Kuka Robotics. Alternatively or in combination, the linkage may comprise a rotating and translating linkage as described in PCT. App. No. PCT/US2015/048695, filed Sep. 4, 2015, entitled “PHYSICIAN CONTROLLED TISSUE RESECTION INTEGRATED WITH TREATMENT MAPPING OF TARGET ORGAN IMAGES”, published as WO 2016/037137, the entire disclosure of which is incorporated herein by reference.

In some embodiments, a user input device 428 is coupled to processor 423. The user input device 428 may comprise any suitable user input device such as a foot pedal, a pointing device, a joystick, a mouse, a touch screen display, or a robotic arm controller, for example. The input device 428 can be configured to selectively control one or more of the treatment probe 450, the arm 442, the arm 444, or the ultrasound probe, for example. The user input device 428 may comprise any suitable number and combination of input devices, and the processor can be configured to allow the user to direct control to any suitable input device.

The patient is placed on the patient support 449, such that the treatment probe 450 and ultrasound probe 460 can be inserted into the patient. The patient can be placed in one or more of many positions such as prone, supine, upright, or inclined, for example. In some embodiments, the patient is placed in a lithotomy position, and stirrups may be used, for example. In some embodiments, the treatment probe 450 is inserted into the patient in a first direction on a first side of the patient, and the imaging probe is inserted into to the patient in a second direction on a second side of the patient. For example, the treatment probe can be inserted from an anterior side of the patient into a urethra of the patient, and the imaging probe can be inserted trans-rectally from a posterior side of the patient into the intestine of the patient. The treatment probe and imaging probe can be placed in the patient with one or more of urethral tissue, urethral wall tissue, prostate tissue, intestinal tissue, or intestinal wall tissue extending therebetween.

The treatment probe 450 and the imaging probe 460 can be inserted into the patient in one or more of many ways. During insertion, each arm may comprise a substantially unlocked configuration such the probe can be desirably rotated and translated in order to insert the probe into to the patient. When a probe has been inserted to a desired location, the arm can be locked. In the locked configuration, the probes can be oriented in relation to each other in one or more of many ways, such as parallel, skew, horizontal, oblique, or non-parallel, for example. It can be helpful to determine the orientation of the probes with angle sensors as described herein, in order to map the image data of the imaging probe to treatment probe coordinate references. Having the tissue image data mapped to treatment probe coordinate reference space can allow accurate targeting and treatment of tissue identified for treatment by an operator such as the physician.

In some embodiments, the treatment probe 450 is coupled to the imaging probe 460. In order to align the treatment probe 450 based on images from imaging probe 460, the coupling can be achieved with the common base 440 as shown. Alternatively or in combination, the treatment probe and/or the imaging probe may comprise magnets to hold the probes in alignment through tissue of the patient. In some embodiments, the arm 442 is a movable and lockable arm such that the treatment probe 450 can be positioned in a desired location in a patient. When the probe 450 has been positioned in the desired location of the patient, the arm 442 can be locked with an arm lock 427. The imaging probe can be coupled to base 440 with arm 444, can be used to adjust the alignment of the probe when the treatment probe is locked in position. The arm 444 may comprise a lockable and movable probe under control of the imaging system or of the console and of the user interface, for example. The arm 444 may comprise a robotic arm, such as a robotic arm comprising 5 to 7 degrees of freedom for example. The movable arm 444 may be micro-actuable so that the imaging probe 440 can be adjusted with small movements, for example a millimeter or so in relation to the treatment probe 450.

In some embodiments the treatment probe 450 and the imaging probe 460 are coupled to angle sensors so that the treatment can be controlled based on the alignment of the imaging probe 460 and the treatment probe 450. An angle sensor 495 is coupled to the treatment probe 450 with a support 438. An angle sensor 497 is coupled to the imaging probe 460. The angle sensors may comprise one or more of many types of angle sensors. For example, the angle sensors may comprise goniometers, accelerometers and combinations thereof. In some embodiments, angle sensor 495 comprises a 3-dimensional accelerometer to determine an orientation of the treatment probe 450 in three dimensions. In some embodiments, the angle sensor 497 comprises a 3-dimensional accelerometer to determine an orientation of the imaging probe 460 in three dimensions. Alternatively or in combination, the angle sensor 495 may comprise a goniometer to determine an angle of treatment probe 450 along an elongate axis 451 of the treatment probe. Angle sensor 497 may comprise a goniometer to determine an angle of the imaging probe 460 along an elongate axis 461 of the imaging probe 460. The angle sensor 495 is coupled to a controller 424. The angle sensor 497 of the imaging probe is coupled to a processor 492 of the imaging system 490. Alternatively, the angle sensor 497 can be coupled to the controller 424 and also in combination.

The console 420 comprises a display 425 coupled to a processor system in components that are used to control treatment probe 450. The console 420 comprises a processor 423 having a memory 421. Communication circuitry 422 is coupled to processor 423 and controller 422. Communication circuitry 422 is coupled to the imaging system 490. The console 420 comprises components of an endoscope 35 that is coupled to balloon 24. Infusion flashing control 28 is coupled to probe 450 to control infusion and flushing. Aspiration control 30 is coupled to probe 450 to control aspiration. In some embodiments, endoscope 21 is coupled to console 420 and the endoscope insertable with probe 450 to treat the patient. Arm lock 427 of console 420 is coupled to arm 422 to lock the arm 422 or to allow the arm 422 to be freely movable to insert probe 450 into the patient.

The console 420 may comprise a pump 419 coupled to the carrier 380 and energy delivery element 200 as described herein.

The processor, controller and control electronics and circuitry can include one or more of many suitable components, such as one or more processor, one or more field-programmable gate array (FPGA), and one or more memory storage apparatuses. In some embodiments, the control electronics controls the control panel of the graphic user interface (hereinafter “GUI”) to provide for pre-procedure planning according to user specified treatment parameters as well as to provide user control over the surgery procedure.

In some embodiments, the treatment probe 450 comprises a balloon 24. In some embodiments, the balloon 24 anchors the distal end of the probe 450 while energy is delivered to energy delivery region 20 with the probe 450. The probe 450 may comprise an energy delivery element 200. In some embodiments, the energy delivery element is located within the balloon 24. Alternatively or in combination, the energy delivery element 200 can be located outside of the balloon 24. In some embodiments the carrier 380 is removable from the linkage and can be replaced with a second carrier 380. For example, a first carrier may comprise a high-pressure nozzle to release a fluid stream for tissue resection with a water jet. Upon completion of the tissue resection, the first carrier 380 is replaced with a second carrier 380. The second carrier 380 may comprise an optical fiber to heat tissue to promote hemostasis, for example. Examples of suitable rapid exchange carriers and probes that can be interchanged while at least a portion of probe 450 remains in the patient are described in U.S. Pat. No. 9,510,852, entitled “Automated image-guided tissue resection and treatment”, issued Dec. 6, 2016, the entire disclosure of which is incorporated herein by reference. In some embodiments, the first carrier comprises a nozzle to release a fluid stream without a balloon over the energy delivery element 200, and the second probe comprises a balloon over the energy deliver element as described herein.

The probe 450 is coupled to the arm 422 with a linkage 430. For example, the linkage 430 can be configured to move the carrier 380 with the energy delivery element 200 carried on the probe in response to instructions on a processor, so as to move the energy deliver element with a desired scan pattern. The energy delivery element 200 may comprise any suitable elements, such as a nozzle to deliver a fluid for tissue resection, one or more electrodes, or an output of an optical fiber.

The linkage 430 comprises components to move energy delivery region 20 to a desired target location of the patient, for example, based on images of the patient. The linkage 430 comprises a first portion 432 and a second portion 434 and a third portion 436. The first portion 432 comprises a substantially fixed anchoring portion. The substantially fixed anchoring portion 432 is fixed to support 438. Support 438 may comprise a reference frame of linkage 430. Support 438 may comprise a rigid chassis or frame or housing to rigidly and stiffly couple arm 442 to treatment probe 450. The first portion 432 remains substantially fixed, while the second portion 434 and third portion 436 move to direct energy from the probe 450 to the patient. The first portion 432 is fixed to the substantially constant distance 437 to the balloon 24. The substantially fixed distance 437 between the balloon 24 and the fixed first portion 432 of the linkage allows the treatment to be accurately placed. The first portion 424 may comprise the linear actuator to accurately position the high-pressure nozzle in treatment region 20 at a desired axial position 418 along an elongate axis of probe 450.

The elongate axis of probe 450 generally extends between a proximal portion of probe 450 near linkage 430 to a distal end having balloon 24 attached thereto. The third portion 436 controls a rotation angle 453 around the elongate axis. During treatment of the patient, a distance 439 between the treatment region 20 and the fixed portion of the linkage varies with reference to balloon 24. The distance 439 adjusts in response to computer control to set a target location along the elongate axis of the treatment probe referenced to balloon 24. The first portion of the linkage remains fixed, while the second portion 434 adjusts the position of the treatment region along the axis. The third portion of the linkage 436 adjusts the angle around the axis in response to controller 424 such that the distance along the axis at an angle of the treatment can be controlled very accurately with reference to balloon 24. The probe 450 may comprise a stiff member such as a spine extending between support 438 and balloon 24 such that the distance from linkage 430 to balloon 24 remains substantially constant during the treatment. The treatment probe 450 is coupled to treatment components as described herein to allow treatment with one or more forms of energy such as mechanical energy from a jet, electrical energy from electrodes or optical energy from a light source such as a laser source. The light source may comprise infrared, visible light or ultraviolet light. The energy delivery region 20 can be moved under control of linkage 430 such as to deliver an intended form of energy to a target tissue of the patient.

The imaging system 490 comprises a memory 493, communication circuitry 494 and processor 492. The processor 492 in corresponding circuitry is coupled to the imaging probe 460. An arm controller 491 is coupled to arm 444 to precisely position imaging probe 460. In some embodiments, the imaging system is configured to view tissue with a resolving power of 100 μm. As used herein, a resolving power refers to the ability to discern two structures from each other.

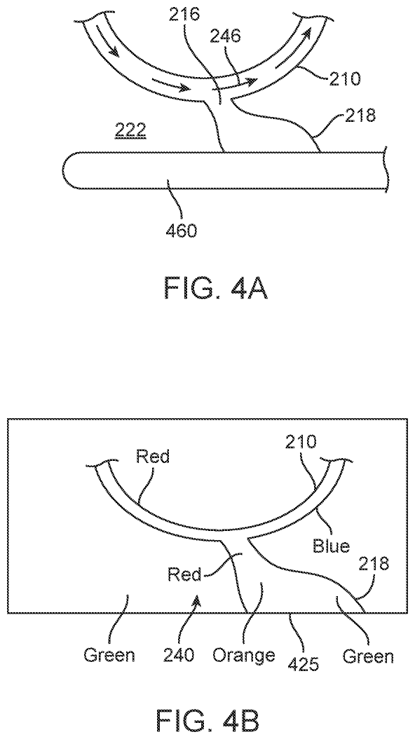

FIG. 4A shows blood flow and a Doppler ultrasound (“US”) probe 460. The Doppler US probe may comprise a TRUS probe as described herein. The Doppler US probe can be configured to detect blood flow toward and away from the probe, including blood from through an opening 216 in the vessel and the resulting plume 218. The plume of blood may form in surrounding fluid 222.

FIG. 4B shows a Doppler ultrasound image 240 on a display 425 for a probe as in FIG. 4A. Blood flow toward the probe is shown in red, and blood flow away from the probe is shown in blue. The Doppler US images can be used to identify bleeding locations, alternatively or in combination with the endoscope as described herein. For example, the processor can be configured with instructions to obtain Doppler US images from the US probe before and after treatment, and identify locations of bleeding in response to changes in blood flow on the Doppler US images. The Doppler US images may comprise two dimensional “2D” or three dimensional “3D” Doppler US images, for example. In some embodiments, the bleeding locations can be identified from blood flow within the vessel and outside the vessel into a cavity, such as a cavity of resected tissue. As shown in FIG. 4B, a plume 218 may form from an opening 216 in the vessel 210. The differing velocities of the blood in the plume may be visualized as different colors. For example, fluid with little or no flow may be indicated as green, such as fluid 222 in the surrounding cavity. Fluid exiting the opening may be indicated as red and as the fluid in the plume slows down, the indicated color may change from orange, representing an intermediate velocity, to green, indicating little to no velocity.

The imaging system can be used to identify the bleeding location in many ways. For example, the processor can be configured to receive to user input to identify bleeding location, or artificial intelligence (“AI”) such as a neural network can be trained to identify the bleeding locations, in response to the US images such as Doppler US images.

In some embodiments, the processor may be configured with instructions to identify the location of bleeding tissue in response to a change in a velocity of a fluid from the Doppler ultrasound image and optionally wherein the fluid comprises blood. The change in velocity may include a decrease in velocity of the fluid along a flow path. In some embodiments, the change in fluid velocity corresponds to a pulsatile flow of the fluid.

In some embodiments, the fluid comprises blood flowing along a blood vessel and the fluid is released through an opening 216 in the vessel wall, such as an opening formed with tissue resection as described herein, e.g. resection with a water jet. The fluid, such as blood, may be released into a second fluid, the second fluid may have a lower velocity than the first fluid and the bleeding location may be identified in response to a change in direction of the fluid through the vessel wall.

In some embodiments, the bleeding location may be identified by registering a first image of the tissue prior to tissue resection with a second image of the tissue after tissue resection. The change in velocity of the fluid may be identified at least in part based on a change between the first image and the second image and optionally the blood vessel of the first image may be measured with a corresponding blood vessel from the second image.

Operator Control, Aiming, Automatic Pattern of Energy Delivery

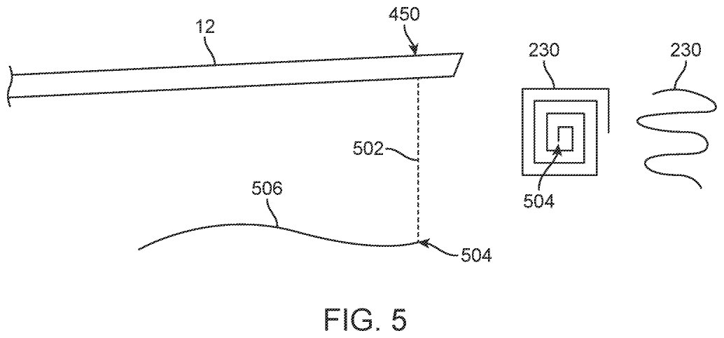

FIG. 5 shows an aiming laser beam 502 on a laser energy delivery probe as described herein. The probe comprises a shaft 12 that can be coupled to a handpiece or a linkage as described herein. In some embodiments, a user input control such as a joystick coupled to the robotic control with linkages as described herein can be used to aim a laser at the target area on a tissue surface 506 for treatment. In some embodiments, aiming is accomplished with a visible laser and the user points the visible beam to the target location 504 with the user input control. Once the aiming beam is positioned at the target location, the user can activate the treatment beam, for example by pressing a foot pedal or button. The treatment beam may comprise visible light energy or substantially invisible light energy such as infrared light energy, e.g. laser energy.

In some embodiments, when the target of interest for the treatment is identified and aligned with the aiming beam, the system mechanically locks in response to user input, so as prevent physician activated movement of robotic structures (e.g. actuators and linkages) so as to allow automated scanning of the treatment laser.

In some embodiments, the method of treatment comprises identifying a central region of a target of interest and determining an area and shape to be treated based on expected underlying anatomy. For example, vessels may approach the surface area of interest at an angle to the treatment probe. Alternately or in combination, the direction of vascularity may be identified using doppler ultrasound.

When the appropriate treatment pattern has been determined, the tissue is treated with an appropriate scan pattern 230. The treatment pattern may comprise any suitable scan pattern 230 such as a circle, oval, an annulus, annuli, or a raster scan pattern for example. The treatment may start at an appropriate distance from the location identified by the user as described herein. For example, the scan pattern may start 3 mm from the identified target location (e.g. a center of interest) and the laser beam scanned in a circular pattern of 6 mm diameter with subsequent smaller and overlapping or not overlapping circles until finishing in the center.

The treatment shape can be sized and dimensioned in many ways. For example a source of bleeding can be identified with a plume 218, and the aiming laser or other pointing device used to identify the target location on an image of the tissue. The treatment pattern may comprise an oval shaped treatment area with the identified target (e.g. the source of bleeding) near the center of one end of the oval (e.g. a first focus of an ellipse) and the extend toward an expected location of underlying vascular anatomy (e.g. a second focus of an ellipse). The distance between the centers can determined based on an expected location of the blood vessel at a desired depth based on a model of vascular anatomy, for example.

Using a Balloon

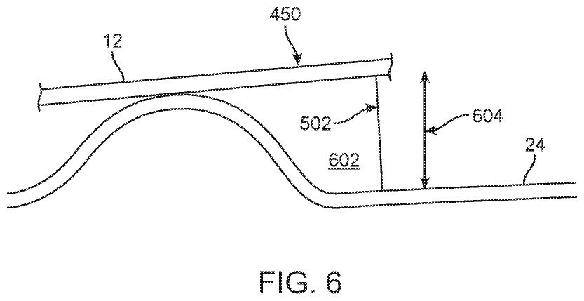

FIG. 6 shows a balloon 24 with a substantially non-attenuating fluid 602 between a laser energy treatment probe 450 that emits a laser beam 602 and a target site of tissue 506. The probe 450 comprises a shaft 12 that can be coupled to a handpiece or a linkage as described herein. The balloon 24 may comprise a compliant balloon or a non-complaint balloon, for example. The compliant balloon may comprise a material which follows the contour of the patient anatomy, such as the tissue 506, when inflated to an appropriate pressure with a fluid. The fluid 602 can distend the balloon 24 such the balloon follows the contours of the resected tissue 506, for example. The balloon can be filled with any suitable fluid such as a liquid or gas, e.g. CO₂, or saline. In some embodiments, the fluid comprises a substantially non-attenuating fluid (e.g. no more than 10% attenuation between from the light energy source to the balloon), or predictably attenuating liquid. In some embodiments, the balloon material is thermally stable at the treatment temperatures and comprises an optically transmissive material. In some embodiments, the balloon comprises a transparent material for at least one wavelength of light to allow visualization of the treatment site. Alternatively or in combination, the balloon can be configured to substantially absorb the treatment energy (e.g. at least 50% of laser energy) so as to localize heating to tissue in proximity to the balloon. In some embodiments, the balloon comprises a material that is radio-frequency transparent and is configured to allow total or near total electromagnetic energy transmission through the balloon into the underlying tissue raising the temperature, such as to promote hemostasis.

The balloon can be configured to gently press against the tissue to provide a more uniform treatment of the underlying tissue. For example, the balloon can press filaments toward the resected tissue surface. In some embodiments, by complying with the patient anatomy, loose tissue is pressed to the underlying tissue shape such as a resected tissue shape, so as to provide a more uniform surface for treatment. For example, remnant filaments (e.g. fluffy tissue) subsequent to water jet resection can be pressed with the balloon toward the resected tissue surface so as to compress the filaments tissue and allow a more direct delivery of laser energy to the underlying tissues to provide hemostasis or other treatment.

In some embodiments, a substantially non-attenuating fluid results in decreased variation in energy delivered to the target tissue at different distances of transmission through the substantially non-attenuating fluid as compared with an attenuating fluid.

In some embodiments, the fluid comprises at least some attenuation of the light energy, such as the light energy in the laser beam 502, by a predictable amount, and the system can be configured to adjust the treatment irradiance in response to the attenuation. For example, an ultrasound imaging system or other imaging system can be used to determine the distance of light energy transmitted through the fluid from the output window to the tissue surface. The distance from emitting probe to tissue surface can be used to determine the power, duration, and motion appropriate to provide hemostasis or other therapy.

With a non-compliant balloon, such as a cylindrical or cigar shaped balloon, which may have a known radius, the balloon can be inflated to achieve a known distance 604 from emitting probe to balloon surface, which can reduce variability of attenuation, as well as variability of angle of incidence of the light energy toward the location of treatment. In some embodiments, blood flow to the tissue is decreased with balloon pressure, which may provide more efficient heating and coagulation within the tissue related to decreased perfusion of the tissue.

Blood Detection and Treatment

In some embodiments, bleeding locations are visible in the images so as to allow identification of the target site. The target site can be identified in many ways, such as with human interface or machine vision identification of bleeding site. In some embodiments, the target site is identified by a user interface with the imaging system or artificial intelligence, such as machine vision. The treatment region is determined automatically and treatment automatically enabled. Alternatively or in combination, the treatment region can be verified by a physician. In some embodiments, the physician determines the treatment region as described herein.

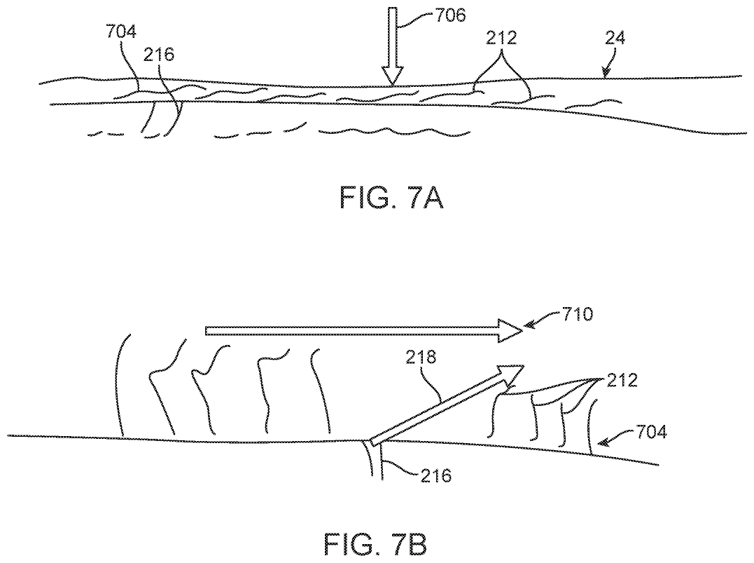

FIG. 7A shows an inflated balloon placed against a tissue resection profile. The inflated balloon can be coupled to a probe comprising a shaft as described herein that can be coupled to a handpiece or a linkage as described herein. The balloon 24 is deployed to contact and apply a compressive force 706 to residual tissue 702, such as residual tissue filaments comprising collagen. In some embodiments, the balloon compresses the residual tissue 702 along the wall of the tissue surface, e.g. the resection profile 704. In some embodiments, the active bleeding source, is visible through the balloon. The aiming laser beam can be directed to the opening of the vessel 210 based on blood in the image, so that the aiming laser beam is directed to the area of visible blood, e.g. substantially centered in the visible area of blood. Although reference is made to a laser beam, another marker can be used to identify the treatment area, such as a computer-generated marker. Using the energy delivery element as a laser pointer, the light energy beam may be coupled to a robotic control element. The laser pointer may be used to identify the source of bleeding. Once the source of bleeding is identified through direct visualization, the location can be identified and marked through a user input device on the console (e.g. point and click at the site of bleeding). Using the rotation of the energy element and axial drive of the shaft, the console may drive the energy source to the source of bleeding and apply energy to achieve hemostasis. In a separate embodiment, the robotically driven element may use the same or separate laser to measure distance to the tissue surface using Lidar or other optical methods such as optical coherence tomography (OCT) in order to ensure precise location of the energy delivery element.

FIG. 7B shows fluid flow 710 over a tissue resection profile 704 and plume of blood 218 shown with an arrow. In some embodiments, the fluid flow 710 is provided with a probe 450 comprising a shaft that can be coupled to a handpiece or a linkage as described herein. In some embodiments with an aqueous environment, the irrigation and evacuation (e.g. aspiration) capabilities of the treatment system as described herein can be used to generate a fluid flow to remove blood from the bleeding location. In some embodiments, a flow of working fluid induces blood to flow to improve access to the ruptured vessel. The flow of the fluid and blood can be oriented in any suitable direction, for example from the proximal endoscope viewing port toward the distal end of the probe near the bladder (toward the right). An evacuation port such as an aspiration port can be used to draw fluid from the endoscope viewing port and laser delivery element to clear the field of view and provide improved visualization. This removal of blood can also help to identify the location of bleeding from the vessel. This flow can allow a physician viewing the target area or vision system to identify the approximate location of bleeding.

Laser Fiber Positioning Relative to Tissue

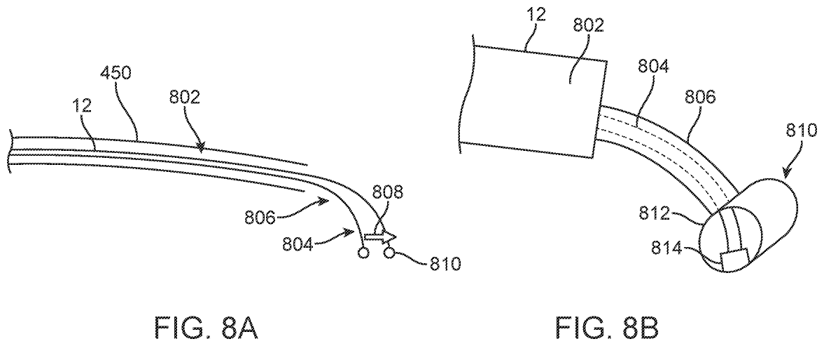

FIG. 8A shows an extendable optical fiber 804 deployed from an end of a lumen 802 of a probe 450. The probe 450 comprises a shaft 12 that can be coupled to a handpiece or a linkage as described herein. The optical fibers 804 can be configured to deflect in a free form configuration, for example when extending from the lumen. The optical fiber can be advanced along the delivery lumen 802 in a substantially straight configuration, and then deflect when extending beyond the delivery lumen. The one or more optical fibers can be advanced and retracted to scan the laser energy along the tissue, such as along the movement direction 808. The one or more optical fibers can be rotated to rotate the scan pattern, for example. In some embodiments, the optical fiber or a material external to the optical fiber comprise a spring-loaded bias so as to deflect the optical fiber extending from the end of the delivery lumen. For example, the optical fiber can be at least partially enclosed within a shaped tube, such as a sheath 806, that deflects in a free-standing configuration, e.g. with a curvature or other suitable deflection. In some embodiments, the length of deployment of the curved tube provides for variable lateral offset. In some embodiments, the optical fiber can be advanced from the distal end of the lumen to contact tissue with deflection and a slight pressure to the tissue. The optical fiber can be advanced further if the tissue wall is located farther from the distal end of the lumen.

In some embodiments, the optical fiber extends along a structure such as a tube with an internal channel, e.g. a lumen, sized to pass an irrigation fluid around the optical fiber within the lumen. The optical fiber and shaped tubular structure can be moved together within the delivery lumen to pass a flushing fluid toward the treatment location. The fluid delivery lumen may contain the optical fiber. Alternatively or in combination, the fluid delivery lumen may comprise a separate lumen from the lumen of the optical fiber. The fluid delivery lumen can be connected to a flushing source as described herein. In some embodiments, the fluid delivery lumen extends coaxially with the laser energy delivery optical fiber. The flushing fluid connected to and in proximity to the optical fiber promotes the presence of clear liquid in the path of the laser beam which provides more accurate delivery of energy to the target tissue. In some embodiments, the fluid provides cooling to the treated tissue and may decrease degradation of the distal end of the optical fiber.

In some embodiments, the optical fiber comprises an opening on the end of the fiber to deliver light energy to the tissue e.g. an end fire optical fiber, although other approaches can be used as described herein.

FIG. 8B shows a tissue engagement structure 810 comprising a roller 812 coupled to an optical fiber 804. In some embodiments, the optical fiber 804 extends at least partially into the tissue engagement structure 810 to deliver light energy to the tissue. The tissue engagement structure 810 may comprise a tissue contact surface dimensioned larger than a transverse cross-section of the optical fiber in order to decrease pressure to the tissue as compared to the optical fiber directly contacting tissue, which can allow the engagement structure coupled to the optical fiber to move more freely along the tissue surface than the optical fiber. The engagement structure 810 may comprise a curved contact surface to allow the engagement structure to move along the tissue, for example to slide along the tissue. The tissue contact surface of the engagement structure may comprise any suitable shape such as a cylindrical shape or a spherical shape, for example. The engagement structure may comprise two wheels adjacent to the fiber or a sphere or cylinder with the optical fiber positioned within. In some embodiments, the tissue engagement structure comprises a roller 814 configured to roll along the tissue surface with the distal end of the optical fiber spaced apart from the tissue. The optical fiber may be configured to deflect when advanced from an opening of a delivery lumen toward tissue. In some embodiments, the one or more optical fibers is at least partially enclosed in a housing, so as to deflect one or more optical fibers when advanced from a distal end of a delivery lumen. The tissue engagement structure can be sized to fit within the delivery lumen, so as to allow insertion and removal of the engagement structure from the tissue treatment site.

The engagement structure can be sized and shaped in many ways. In some embodiments, the engagement structure comprises a surface comprising a dimension across within a range from 2 to 10 mm and optionally within a range from 3 to 7 mm. In some embodiments, the engagement surface comprises one or more of a curved surface, a flat surface, an inclined surface or a bevel to allow the engagement structure to slide along a resected tissue with filaments.

The one or more optical fibers can be moved by the surgeon moving a handle coupled to a proximal portion of the optical fiber, so as move the distal tip of the optical fiber with rotational and translational movement as described herein. For example, deployment of this apparatus could be via direct physician manipulation with an external handle providing both axial in and out motion as well as radial angular positioning to position the optical fiber for treatment. In some embodiments, the handle coupled to the optical fiber is configured to provide treatment of a full 360-degree rotation and translation of any suitable length.

Alternatively or in combination, the optical fiber can be moved with a linkage under computer control as described herein. In some embodiments with robotic control of the position of the end of the optical fiber, a proximal portion of the optical fiber is coupled to an apparatus, e.g. a linkage, which provides for accurate positioning of the distal end of the optical fiber relative to the target tissue. The linkage may provide rotational and translational movement as described herein. The engagement structure can be moved similarly with the distal end of the optical fiber.

In some embodiments, the user can input target locations for treatment. For example, the user such as a physician can input target locations based on images shown on a display. In some embodiments, treatment locations can be determined based on mapping or predictive anatomy from a tissue resection profile, such as water jet resection. Alternatively or in combination, ultrasound images and endoscopic camera images can be used. In some embodiments, the processor comprises instruction to determine the target region to be treated with artificial intelligence algorithms such as machine vision.

Laser Energy Delivery—Laser Energy Distribution Via Lenses and Mirrored Surfaces

In some embodiments, an optical structure is coupled to the optical fiber near the end of the optical fiber to provide a desired distribution of light energy to the tissue. The optical structure can be configured to provide beneficial distribution of light energy to the treated surface. The light from the exit aperture of the probe generally diverges toward the tissue so that the irradiance at the tissue surface may be lower than near the probe. In some embodiments, the approach provides a more uniform distribution of light energy delivered to the treated tissue. In some embodiments, the processor is configured with instructions to determine the distribution of light energy to the tissue in response to the distance to the tissue and the treatment time and movement of the probe to determine the desired treatment, e.g. hemostasis. The flow of fluid around the tissue may be taken into consideration in determining the treatment time. In some embodiments, there is relatively little fluid flow from urine or a flushing fluid and the treatment can be determined accordingly. Alternatively or in combination, fluid flow can be provided by urine or a flushing fluid as described herein, in which the irradiated tissue is cooled at least partially by fluid flow, e.g. convection. The time of treatment and scanning pattern can be determined in response to the fluid flow with other parameters as described herein, e.g. distance and divergence.

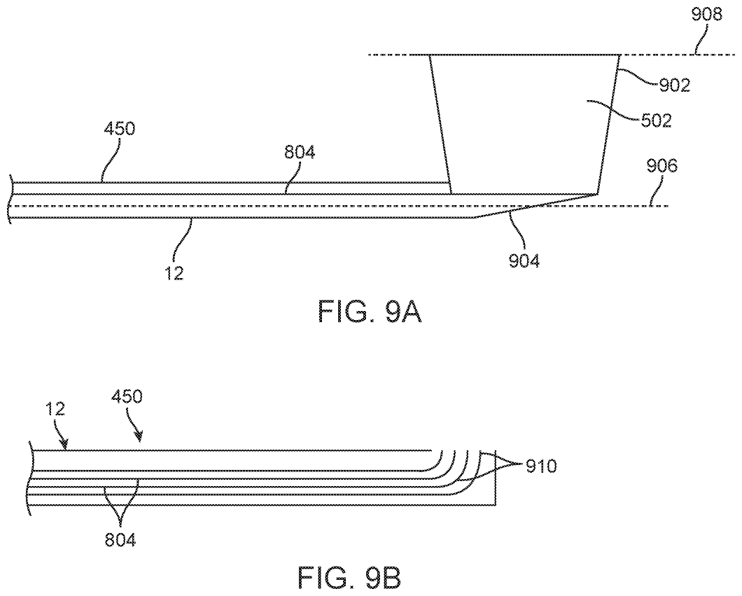

FIG. 9A shows a side emitting laser energy delivery probe 450. The probe 450 comprises a shaft 12 that can be coupled to a handpiece or a linkage as described herein. In some embodiments, a conical cut fiber 804 with reflective surface 904 is configured to direct light energy from the side of the optical fiber 804 toward tissue. The side emitting laser probe can be configured to emit laser energy with an elongate cross-section, such that the beam 502 irradiating tissue comprises an elongate cross-section. The elongate cross-section 902 of the beam 502 can be one or more of rotated or translated to treat a surface area of tissue. The elongate beam can allow the beam to irradiate an increased area of tissue, which can be helpful for scanning the beam and decreasing the amount of time to cover an area of tissue as opposed to a beam focused to a point. The reflective surface 904 can be sized and shaped in many ways and may comprise a cylindrical or parabolic profile, for example. The reflective surface may comprise a mirror surface with a coating, for example. In some embodiments, the mirror surface comprises a backing to support the coating. The mirror can be configured to direct light energy along an elongate pattern such as a line. Alternatively or in combination the reflective array can be configured to provide an array of overlapping beams of energy, such as a linear array of overlapping beams of light energy directed to the tissue. Alternatively, a lens system which selectively directs a desired percentage of energy per linear length of the end fiber and directs it in a line toward the tissue. In some embodiments, the lens system comprises an end lens system comprising one or more lenses that can be manipulated with the fiber. In some embodiments, this approach allows targeting a surface area and treatment surface with one or more of rotational movement or translational movement of the laser. In some embodiments, the elongate axis 906 of the fiber may be substantially parallel to the elongate axis 908 of the elongate beam 502, for example to within about 10 degrees of parallel.

FIG. 9B shows a probe 450 comprising a plurality of optical fibers 804 such as an array of optical fibers to deliver laser energy to tissue. The probe comprises a shaft 12 that can be coupled to a handpiece or a linkage as described herein. The plurality of optical fibers 804 can be arranged with a plurality of ends 910 of the optical fibers oriented to direct light energy to tissue. In some embodiments, the plurality of optical fibers is arranged to provide an elongate beam as described herein. In some embodiments, the plurality of optical fibers 804 comprises a linear assembly of a plurality of end firing fibers to provide an array of laser energy delivered to the tissue. The array of optical fibers 804 can be configured in many ways to provide a desired energy distribution to tissue. In some embodiments, the ends of the optical fibers are arranged to at least partially overlap the laser beams. The plurality of fibers can be coupled to a single laser source or to a plurality of laser sources to provide the desired energy density. For example, a single laser source can be coupled to a plurality of optical fibers with a fiber optic beam splitter. Alternatively, a plurality of optical fibers can be coupled to a plurality of laser sources, in which each laser source is coupled to an optical fiber, for example. In some embodiments, the plurality of optical fibers is coupled to a plurality of optical structures, such as one or more of lenses, prisms, or mirrors to direct the light energy to the tissue with an energy distribution.

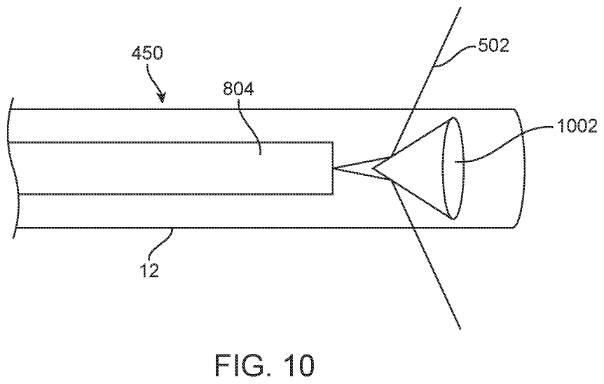

FIG. 10 shows an optical fiber 804 coupled to a conical mirror 1002. The probe 450 comprises a shaft 12 that can be coupled to a handpiece or a linkage as described herein. Laser energy from an end fire optical fiber 804 is spread by a conical structure 1002 into an area comprising a shape corresponding to at least a portion of an annulus. The conical structure may comprise a conical portion configured to distribute light in a 360 degree annular pattern. Alternatively, the annular structure may comprise a portion of a cone such as a ⅓ or half cone configured to provide a sided delivery of the energy. This approach would allow targeting an area and treat it with one or more of a linear movement or a rotational movement of the laser beam emitted from the probe.

Although reference is made to a laser beam, the light energy emitted from the probe may comprises light energy from any suitable source such as high energy flashbulb, for example. The probes described with reference to FIGS. 9A to 10 can be combined with other treatments as described herein, such as with a balloon, fluid flow, or robotic movement, and combinations thereof, for example.

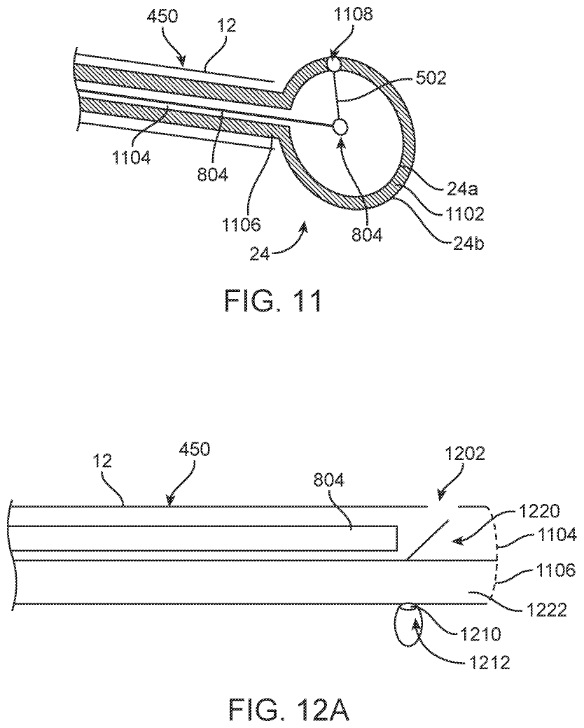

FIG. 11 shows a double balloon 24 comprising an inner balloon 24a and an outer balloon 24b configured to define a fluid flow channel 1102 between the inner balloon and the outer balloon. The probe 450 comprises a shaft 12 that can be coupled to a handpiece or a linkage as described herein. In some embodiments, a source of laser energy 502 such as an optical fiber 804 is positioned within a dual layer balloon 24, which provides for laser radiation penetration to the target tissue through both layers of balloon material at a heating point 1108. The channel 1102 extending between the two layers can provide cooling to the tissue in contact with the balloon so as to decrease thermal damage to adjacent tissue during treatment. The inner balloon 24a can be filled with a first fluid, such as a gas or liquid, and the second balloon 24b can be filled with a second fluid, such as a liquid, so as to provide cooling to the tissue. The fluid in the channel between the inner balloon and the outer balloon may comprise a chilled fluid, for example. In some embodiments, the inner balloon is filled with a gas and the channel between the inner balloon and the outer balloon is filled with a material comprising a heat capacity greater than the gas, such as a liquid, gel, or other suitable material.

In some embodiments, the probe comprises a first lumen 1104 to provide fluid to an interior of the balloon inside the first layer and a second lumen 1106 to provide a liquid to the channel. In some embodiments, the fluid to the interior inside the first layer of the balloon comprises a gas, and the fluid in the channel comprises a liquid.

Combination Water Jet and Laser Probes

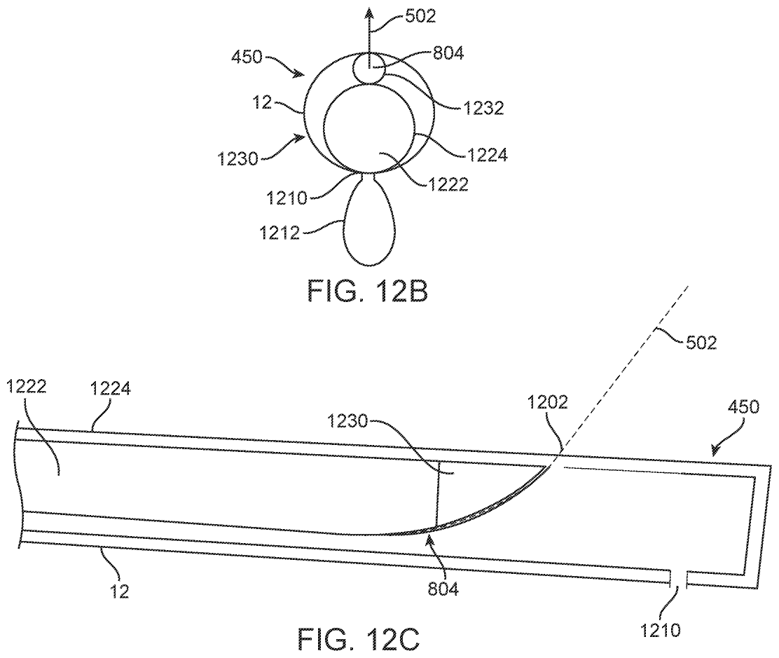

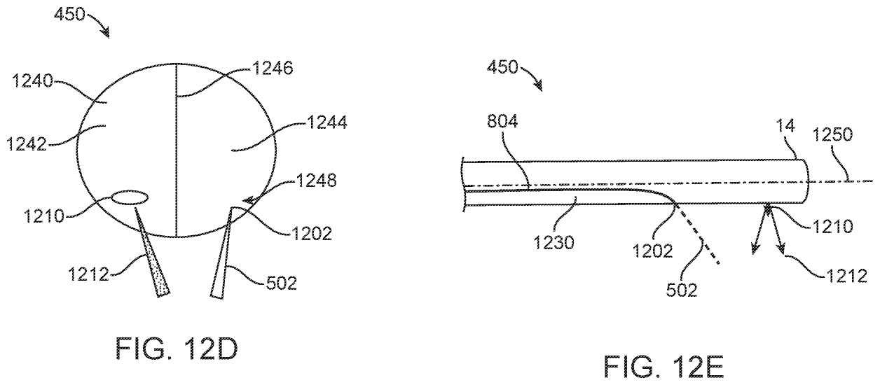

FIG. 12A shows a combination treatment probe 450 comprising an optical fiber 804 to release energy to the tissue at a first location on the probe and a nozzle 1210 to release a water jet 1212 at a second location on the probe. The probe 450 comprises a shaft 12 that can be coupled to a handpiece or a linkage as described herein. In some embodiments, the first location 1202 of the probe to release light energy is located on a first side 1104 of the probe 450 that is opposite a second side 1106 of the probe from the nozzle 1210 to release fluid such as a liquid. The probe can be used to treat tissue with the water jet 1212 and then rotated at least 90 degrees, e.g. at least 150 degrees, to treat tissue with the light energy. Alternatively, the water jet nozzle and source of laser energy can be located on the same side of the probe. In some embodiments, the probe comprises an optical fiber extending to an end configured to direct energy toward tissue or an appropriate optical structure 1220 as described herein, such as one or more of a mirror, a prism, a lens or a conic structure, and then, in some embodiments, out an aperture 1202. Alternatively or in combination, the optical fiber may comprise a bent optical fiber as described herein. The probe may comprise an internal lumen 1222, for example a lumen of a tube 1224, configured to provide pressurized liquid such as water to the nozzle. In some embodiments, the optical fiber extends along the probe with the optical fiber outside the high-pressure tube 1224 coupled to the nozzle, for example in an adjacent substantially parallel configuration, in which the optical fiber extends alongside the water jet tube. Alternatively, the probe may comprise the high-pressure tube with the optical fiber extending within the high pressure tube, and the optical fiber extending through an aperture and sealed.

FIG. 12B shows a treatment probe 450 comprising a nozzle 1210 coupled to a pressurized jet lumen 1222, which delivers a water jet to tissue, and an optical fiber outside the pressurized lumen. The probe comprises a shaft 12 that can be coupled to a handpiece or a linkage as described herein. In some embodiments, a tube defines the high-pressure water jet lumen 1222 and the optical fiber extends outside the tube. The optical fiber may be enclosed within a sheath 1232 that extends along the outside of the high-pressure tube, for example in an adjacent configuration. In some embodiments, the optical fiber and the high-pressure tube are enclosed within an elongate structure such as an elongate tube 1230.

FIG. 12C shows a high-pressure lumen 1222 coupled to a nozzle 1210 to release a water jet and an optical fiber 804 to treat tissue with light energy, in which the optical fiber is located in the high-pressure lumen 1222. The probe 450 comprises a shaft 12 that can be coupled to a handpiece or a linkage as described herein. In some embodiments, optical fiber 804 is coupled to a sealed exit portal or aperture 1202 to decrease leakage from the high-pressure lumen. For example, the structure defining the high-pressure lumen may comprise an aperture sized to receive the optical fiber. The optical fiber can be coupled to the aperture with a friction fit, a compression fit, or an adhesive, and combinations thereof, for example. The optical fiber may comprise a bent optical fiber, and the bent optical fiber may comprise a support structure 1230 with a bend radius, for example as described in U.S. application Ser. No. 16/362,316, filed Mar. 22, 2019, entitled “Tissue treatment probe with bent optical fiber”, published as US 2019-0216485, the entire disclosure of which is incorporated herein by reference.

FIG. 12D shows a probe 450 comprising a light source 1248, such as an optical fiber, and a nozzle 1210 at different locations on a probe 450. In some embodiments, the light source 1248 and nozzle 1210 are oriented so as to at least partially overlap at a tissue location. Alternatively, the nozzle 1210 and light source 1248 can be configured in a non-overlapping arrangement and the probe configured to move to resect tissue with the fluid stream 1212 such as a water jet with first probe movements, and to move to treat tissue with light to decrease bleeding with second movements. In some embodiments, the probe comprises a midline 1246 extending along an elongate axis 1250 of the probe, and the light source and nozzle are located on opposite sides of the midline, for example on a first side 1242 of the midline 1246 and a second side 1244 of the midline 1246. In some embodiment, the lumen comprises a high-pressure lumen extending along the first side and the second side of the probe, in which the lumen is fluidically coupled to the nozzle. An optical fiber 804 may extend along an interior of the lumen, for example along at least a second side of the lumen to an output port, such as a sealed aperture 1202 or a window to release light energy with a light beam 502 as described herein. The nozzle 1210 and light source 1248 may be inclined in relation to the midline, in order to direct the fluid stream such as a water jet toward the light beam. In some embodiments, the light beam and fluid stream are oriented so as to overlap along the midline at a distance from the probe.

FIG. 12E shows a probe 450 comprising a light source, such as optical fiber 804, and a nozzle in which the light source and the nozzle are space apart axially. The probe 450 comprises an optical fiber coupled to an output port and a lumen such as a high-pressure lumen coupled to a nozzle. An optical fiber 804 extends along the probe to an output aperture 1202 such as a sealed aperture in a tube defining the lumen. In some embodiments the optical fiber extends along the lumen fluidically coupled to the nozzle 1210. One or more support structures within the lumen can be configured to support the optical fiber with a bend within the lumen, for example. Alternatively or in combination, the optical fiber may extend along a separate lumen or channel of the probe that is spaced apart from the lumen coupled to the nozzle. In some embodiments, the probe comprises a distal end 14, and the nozzle is located proximally to the distal end. In some embodiments, the light aperture in the probe to emit light is located proximally to the nozzle. Alternatively the light source can be located distally to the nozzle.

The light beam can be emitted from the probe at any suitable angle. In some embodiments, the light beam is emitted from the probe at an oblique angle relative to the elongate axis 1250 of the probe, for example so as to provide oblique illumination as described herein. Alternatively or in combination, the light beam 502 can be emitted from the probe at an angle that is substantially perpendicular to an elongate axis of the probe, for example within approximately 15 degrees of perpendicular. In some embodiments, the light beam 502 is emitted at an angle to the elongate axis 1250 so as to overlap with the fluid stream, e.g. water jet, at a distance from the probe. In some embodiments, the light source comprises an optical fiber extending along the probe and wherein the optical fiber comprises a bend relative to an elongate axis of the probe in order to direct a light beam to the tissue at a non-parallel angle to the elongate axis of the probe. Alternatively, the optical fiber may comprise a bend of approximately 90 degrees to direct the beam at angle of approximately 90 degrees to the elongate axis of the probe.

The probe comprising the light source and the nozzle can be configured in many ways. In some embodiments, the probe is configured to rotate about the elongate axis of the probe and to translate along the elongate axis. The first location and the second location are located along the probe at spaced apart locations and a similar rotational angle with respect to the elongate axis, and the probe is configured to translate the light source along the elongate axis to treat the region of tissue treated with the water jet.

In some embodiments, the nozzle is aligned relative to an elongate axis of the shaft to direct the water jet to a first region of tissue, and the light source is aligned relative to the elongate axis to direct the light beam to a second region of tissue overlapping with the first region when the nozzle is directed toward the first region.

In some embodiments, the shaft comprises a first side and a second side and wherein first side comprises the first location and the second location. In some embodiments, a midline of the probe separates the first side and the second side.

In some embodiments, the nozzle to emit the fluid stream and light source are located along a midline of the probe and spaced apart axially. In some embodiments, the nozzle is located distally and the light source on the probe, e.g. optical fiber end, located proximally to the nozzle. With this configuration tissue can be resected with the water jet, and the probe subsequently advanced distally to coagulate the resected tissue with the light beam. Alternatively, the light source can be located distal to the nozzle and the probe retracted proximally to treat resected tissue with the light beam.

In some embodiments, the light beam and fluid stream are configured to substantially overlap at a distance from the probe so as to allow substantially simultaneous treatment with the water jet and light beam. Alternatively or in combination, the light beam can be used to treat resected tissue with the light beam shortly after treatment with the water jet, for example within a few seconds of rejection with the water jet.

In some embodiments, the probe comprising the optical fiber to emit a laser beam and high pressure lumen to release as water jet from a nozzle is coupled to a robotic linkage configured to resect tissue with the water jet and to coagulate tissue with the light energy from the optical fiber, such as laser energy from the optical fiber. A robotic linkage may be coupled to any treatment source, such as any energy source, and the robotic linkage can be used for imaging, treatment, distance measurement, cautery, or some other purpose or combination of purposes.

Oblique Angle of Incidence

While the laser probe can be configured in many ways, in some embodiments, the probe is configured to direct light energy toward tissue with an oblique angle of incidence, for example an oblique angle of incidence with respect to the surface of the tissue such as a surface of resected tissue. In some embodiments, light energy is transmitted to an underlying vessel to coagulate the vessel away from the vessel opening. This approach can have the benefit of decreasing obscuration of the light energy by blood located near the bleeding vessel. Although reference is made to an oblique angle with respect to the tissue surface, the oblique angle may comprise an angle within a range from about 15 degrees to about 75 degrees, for example within a range from about 30 degrees to about 60 degrees.

Any of the probes described herein can be configured to emit light with an oblique angle of incidence. The oblique angle of incidence can be configured with the direction of flow and endoscope view to improve visibility and decrease obscuration of an underlying blood vessel. In some embodiments, the oblique angle of incidence and flow of the flushing fluid can be at least partially aligned in order to direct light in a direction similar to the direction of flow of the flushing fluid. For example, the probe opening coupled to the source of flushing fluid can be located proximally to the probe opening coupled to the evacuation lumen, e.g. the aspiration lumen, and the probe can be configured to direct light from the probe distally, such that the light beam propagates distally and radially outward and the flushing fluid flows distally from the probe. The endoscope viewing port can be configured to view distally from the probe, in order to view the tissue through the flushing fluid and displace blood away from the endoscope viewing port. Alternatively, the configuration can be reversed, such that fluid flows proximally, the evacuation port is located proximal to the flushing port, and the endoscope viewing port is oriented proximally.

FIG. 13 shows an oblique angle of incidence of laser energy, such as a light beam 502, with respect to a tissue resection profile 704 to delivery laser energy to a blood vessel 210 beneath the tissue resection profile. In some embodiments, the ruptured blood vessel 210 has underlying vascular structure which is substantially intact and undamaged and located away from the tissue resection surface and the opening to the vessel. This substantially undamaged portion of the vessel can be coagulated to decrease bleeding. The portion of the underlying blood vessel located beneath the resected tissue surface and tissue fibers 212 can be treated with decreased interference from blood released from the ruptured vessel, for example. Work in relation to the present disclosure suggests that blood vessels beneath the surface extend at an oblique angle to the surface, and oblique illumination of the vessel relative to the surface can provide illumination transverse to the vessel and may provide improved coagulation of the vessel.

The laser beam 502 can be configured in many ways to treat a blood vessel 210 below the tissue surface 704. In some embodiments, tissue scatters longer wavelengths of light less than shorter wavelengths of light, and the light source can be configured to emit light at an appropriate wavelength. Work in relation to the present disclosure suggests that light comprising a wavelength within a range from about 500 nm to about 600 nm can provide suitable penetration and absorbance of hemoglobin, although other wavelengths can be used. In some embodiments, the tissue penetration depth extends to about 10 mm. The tissue irradiance and duration can be configured to provide coagulation at a depth in the tissue. In some embodiments, the underlying vessel is targeted with the laser beam, for example with endoscopic visualization of the underlying vessel. The laser energy incident at an angle with the tissue can provide improved visualization and penetration of light from the surface of the target tissue, for example by viewing and targeting the tissue around the source of blood, e.g. the ruptured blood vessel. In some embodiments, the blood vessel is treated with a beam with an elongate cross-section as described herein, although other beam shapes can be used, such as a scanning circular spot. The oblique illumination and targeting of the blood vessel can provide the advantage of decreasing damage to adjacent tissue, such as thermal necrosis, by irradiating the blood vessel with decreased interference from blood, for example. Work in relation to the present disclosure also suggests that with the oblique illumination, the blood vessel can be oriented with an elongate axis of the blood vessel that is within about 45 degrees of perpendicular to the beam, which can provide a more localized coagulation of the blood vessel.

The light energy used to treat tissue can be generated in many ways. In some embodiments, a laser is used to generate the light beam. The laser may comprise any suitable laser such as one or more of a gas laser, a liquid laser, a liquid dye laser, a solid-state laser, diode laser, a frequency doubled laser, a frequency mixed laser, a mode locked laser, or a diode pumped laser, for example. The laser may comprise a pulsed laser or a continuous laser. In some embodiments, the laser is coupled to an optical fiber that extends along the probe to direct energy to tissue as described herein. The laser can be configured to emit any suitable wavelength of light, such as one or more of ultraviolet, visible, or infrared light. In some embodiments, the laser comprises a pulsed Nd:YAG laser configured to emit light at 1064 nm, for example. Work in relation to the present disclosure suggest that in some tissues light of approximately 1000 nm has a penetration depth of approximately 1 cm in tissue, which can be well suited for use with oblique illumination or other suitable illumination as described herein.

Toroidal Balloon with Central Laser Approximating the Treatment

FIG. 14 shows a balloon 24 extending around a light energy delivery port 1202 of an optical fiber delivery probe 450. The probe 450 comprises a shaft 12 that can be coupled to a handpiece or a linkage as described herein. The toroidal balloon 24 may comprise a compliant or rigid toroidal balloon. The balloon can be coupled to an inflation lumen as described herein. The laser beam 502 can be emitted from the probe with an appropriate configuration as described herein, for example with a laser beam oriented substantially perpendicular to the axis of the probe. The balloon 24 can extend over the aperture 1202 or window of the elongate shaft and receive laser beam energy. The balloon may comprise a material transparent to the laser beam or an opaque material. The physician can manipulate the probe, for example with a proximal handpiece over a treatment area. Alternatively or in combination, a robotic linkage can be used to move the laser beam over a treatment area as described herein before, during, or after a treatment. In some embodiments, the balloon and laser beam are configured to move together with the probe. For example, the elongate probe shaft can be configured to rotate and translate and the balloon and laser beam rotate and translate with the movement of the probe. Alternatively or in combination, the balloon 24 may allow movement 1404 of the laser beam 502 and probe 450 while the balloon engages the tissue surface 1406 and the resection profile 704 and remains substantially fixed on the tissue surface 1406, for example with translational and rotational movement of the probe while the balloon engages the tissue surface 1406. In some embodiments, the probe comprises an optical fiber 804 that translates and rotates with the balloon and the laser beam. In some embodiments, the balloon is coupled to the probe so as to provide translational movement of the probe and balloon while the probe rotates freely with respect to the balloon, for example with a coupling that allows rotational movement of the probe relative to the balloon. The probe can be moved so as to direct the laser beam to a desired location as described herein, for example with programmed movement or manual manipulations.

In some embodiments, the balloon is configured to change shape as the balloon moves over tissue surfaces 1406 with translational movement, which can be helpful for displacing material such as blood and clots in order to improve visibility of target tissue, such as a blood vessel.

The balloon such as a toroidal balloon can be configured for advancement into a lumen in a narrow profile configuration and expanded to a wider profile configuration when inflated into the lumen. In some embodiments, the probe is inserted into the urethra in a narrow profile configuration and expanded to a larger profile configuration with the balloon placed within one or more of an external sphincter, a prostate P or a bladder neck. The balloon can be advanced and retracted along the interior of the surgically resected space with a substantially constant volume and deformation of the balloon, for example with a compliant balloon.

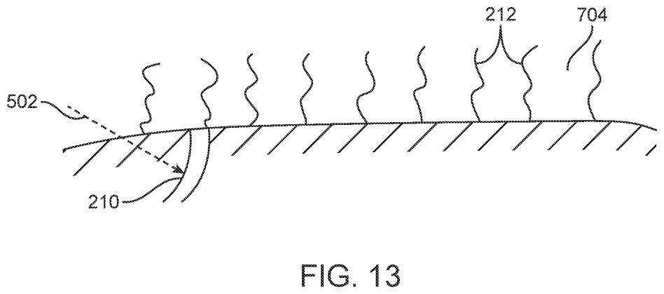

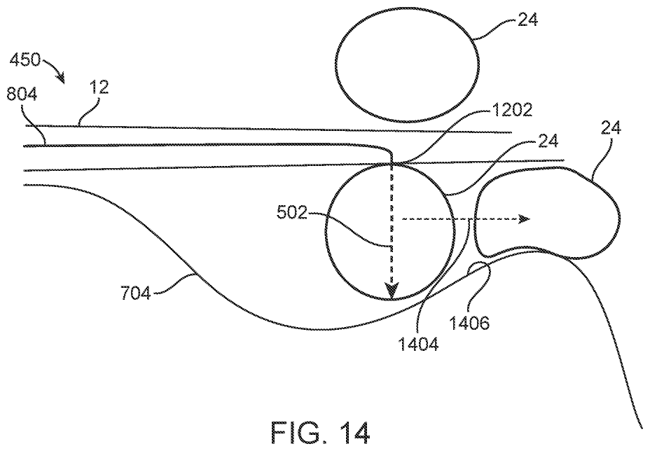

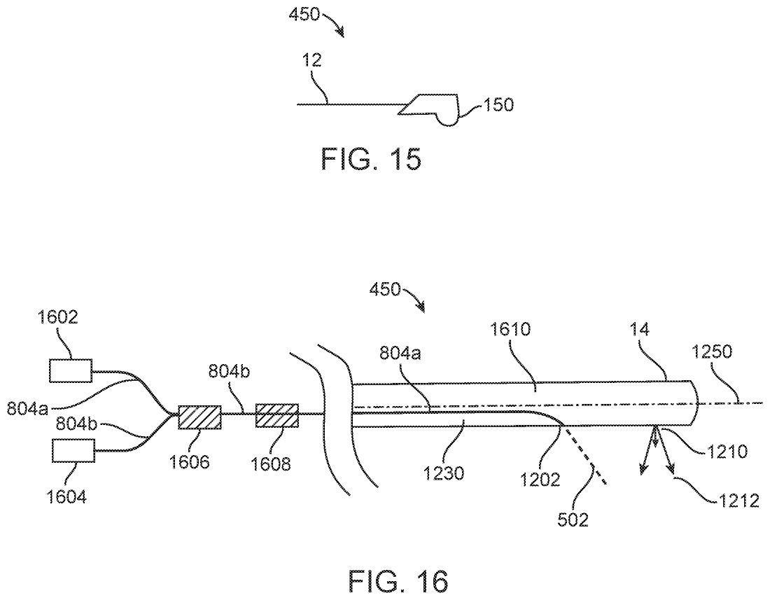

FIG. 15 shows probe 450 comprising a shaft 12 and one or more electrodes 1502, suitable for incorporation with the present disclosure. The one or more electrodes 1502 may comprise any suitable number of electrodes for performing electrocautery. The electrode 1502 may comprise one or more of a monopolar electrode, a unipolar electrode, or a bipolar electrode, or an electrode array, for example. The electrode 1502 can be sized and shaped in many ways, and may comprise one or more of a button electrode or a loop, for example. The shaft 12 can be coupled to a handle and moved manually or coupled to a linkage and moved under processor control as described herein, and combinations thereof. In some embodiments, a location of bleeding tissue is identified as described herein, and the electrode 1502 is moved to the bleeding location in response to processor commands to cauterize the tissue at the bleeding location. The probe 450 can be combined with one or more probes as described herein, for example to provide fluid flow and visualization of the bleeding region of tissue, and to identify the tissue to be treated, for example.

FIG. 16 shows a probe 450 comprising a nozzle 1210 and an optical fiber 804 in a lumen 1610 configured to treat tissue with a water jet 1212 and coagulate tissue with a treatment beam and aim the treatment beam with an aiming beam. The probe 450 may comprise one or more structures of the probe of FIG. 12E and can be used similarly. The light beam 502 may comprise the treatment beam and the aiming beam for example. The optical fiber 804 of the probe is coupled to a treatment laser 1602 and an aiming laser 1604 with an optical fiber (“OF”) coupler 1606. Additional couplers 1606 and connectors 1608 can be used. In some embodiments, a connector 1608 is configured to connect to the optical fiber of the probe 804a between the optical fiber coupler 1606 and the optical fiber from the probe 804b, which allows the probe to be connected to the treatment laser 1604 and aiming laser 1602 and then removed from the system, for example. In some embodiments, the OF coupler 1606 and connector are located on a console and the optical fiber 804a from the probe is connected to connector 1608 at the console, for example. Although reference is made to an aiming laser beam, any suitable light source may be used to aim the light beam such as a light emitting diode.

The light of the aiming light source 1604, e.g. laser, may comprise any suitable wavelength within a range from about 380 nm to about 800 nm, for example. The treatment laser 1602 may comprise any suitable wavelength to treat tissue to decrease bleeding and may comprise any suitable wavelength, such as an ultraviolet, visible or infrared wavelength. In some embodiments, the aiming laser beam comprises a first wavelength and the treatment laser beam comprises a second wavelength, in which the first wavelength is different from the second wavelength, e.g. non-overlapping wavelengths.

In some embodiments, the aiming laser is activated for the user to aim the optical fiber at the source of bleeding tissue as described herein, and the treatment laser is activated to treat tissue at or near the bleeding location as described herein, for example with a scan pattern as described herein.

The probe can be used in many ways. In some embodiments, the probe is coupled to a handpiece for manual use. Alternatively or in combination, the probe can be coupled to a linkage and moved in response to processor instructions as described herein.

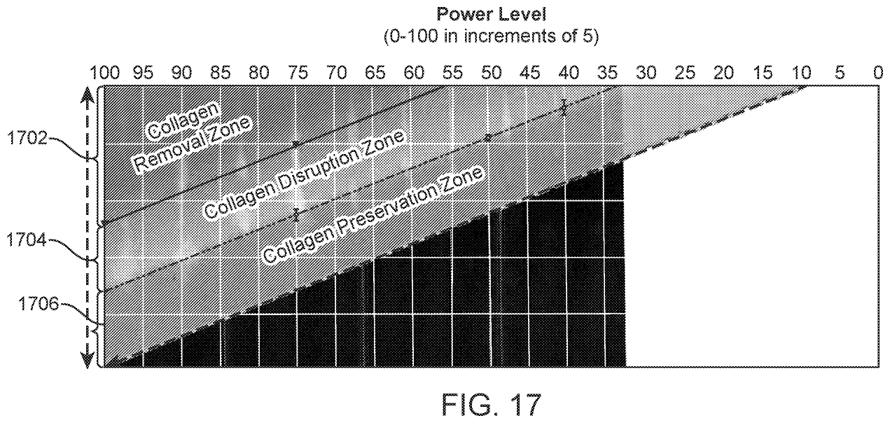

FIG. 17 shows tissue resection zones in accordance with some embodiments. Collagenous tissue such as blood vessels and connective tissue can be more fibrous and have greater strength than softer tissues. In some embodiments, the intensity of the water jet decreases with distance so as to remove different types of tissue with increasing distance from the nozzle. The tissue removal zones may comprise one or more of a collagen and soft tissue removal zone 1702, a collagen disruption and soft tissue removal zone 1704, or a collage preservation and soft tissue removal zone 1706. The selective tissue resection zones may comprise a collagen disruption zone in which collagen is disrupted and substantially remains while soft tissue has been removed, and a collagen preservation zone in which collagenous tissue is substantially preserved and soft tissue removed. The power level of the water jet corresponds to flow rate through a nozzle. As the flow rate increases the power level increases. For lower amounts of power corresponding to lower amounts of flow through the nozzle, no tissue is removed. For increased amounts of power corresponding to the collagen preservation zone, collagen is preserved and soft tissue is removed. For increased amounts of power corresponding to increased flow through the nozzle, collagen is disrupted and soft tissue is removed. In some embodiments, the collagenous fibers described herein correspond to the collagen disruption zone. For further increased power collagen, including the fibers, is also removed.

Although the water jet tissue resection can be configured in many ways, in some embodiments, the high velocity jet causes the tensile disassociation and mechanical lysing of cellular matrix on targeted tissues, such as soft tissues. At short distances corresponding to higher jet velocities (e.g. collagen removal zone) the tissues are broken to small fragments and distributed into the surrounding environment and evacuated as described herein. At greater distances from the nozzle the jet velocity decreases, and the selectivity becomes apparent showing tissues of lower tensile strength disassociated leaving higher tensile strength materials enduring the treatment and remaining attached. In some embodiments, with water jet tissue resection collagen fibers comprising a white cotton like tissue remains, which is visible in the surgical space and can be referred to fluffies.

The tissue treated with the water jet may comprise one or more of fibers (elastic and collagenous fibers), ground substance and cells. Ground substance is primarily composed of water and large organic molecules, such as glycosaminoglycans (GAGs), proteoglycans, and glycoproteins.

In some embodiments, these remaining tissue fibers correspond to collagenous fiber components of the original cellular support structure and blood vessels. For example, the tissue resected may comprise cellular tissue held together with supporting tissue fibers, such as reticular fibers. Without being bound by any particular theory, in some embodiments the collagen fibers remaining after tissue resection comprise reticular fibers from which cells have been removed. In some embodiments, the reticular fibers comprise reticulin, which is a type of fiber located in connective tissue and composed of type III collagen secreted by reticular cells. Reticular fibers can crosslink to form a fine meshwork, e.g. reticulin. In some embodiments, this network acts as a supporting mesh in soft tissues such as liver, bone marrow, glandular prostate tissue and the tissues and organs of the lymphatic system.

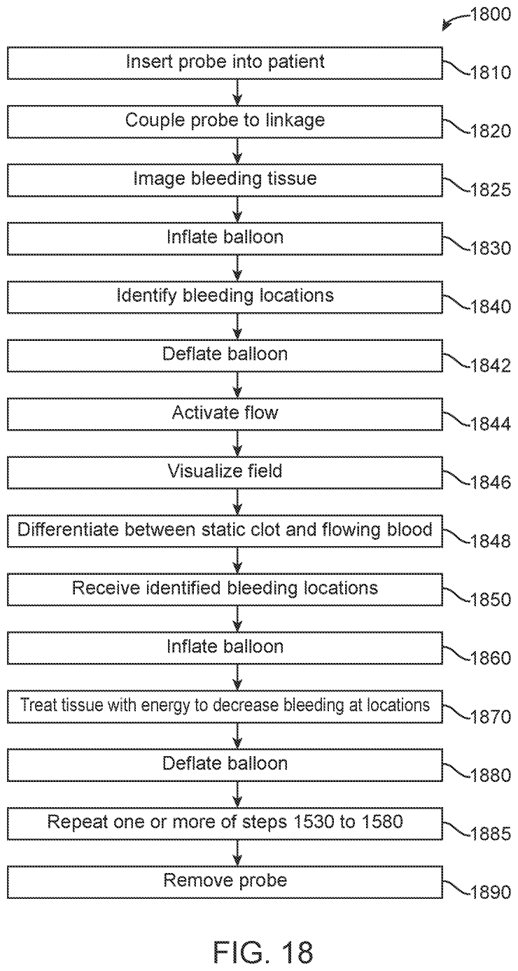

FIG. 18 shows a method 1800 of reducing bleeding of a patient. The method 1500 can be used at any suitable location, such as a surgical site of a patient

At a step 1810, a probe is inserted into patient. The probe may comprise any suitable probe, such as a probe described herein.

At a step 1820, the probe is coupled to linkage as described herein.

At a step 1825, the bleeding tissue is imaged. The bleeding tissue can be imaged in one or more of many ways as described herein. For example, the bleeding tissue can be imaged with one or more of an endoscope, an ultrasound probe, or a Doppler ultrasound probe, such as a transrectal Doppler ultrasound probe.

At a step 1830, a balloon is inflated. The balloon may comprise any suitable balloon as described herein, such as a compliant or a non-compliant balloon configured to engage the tissue. In some embodiments, the engagement of the balloon with the tissue during inflation is imaged, for example to determine snugness of the fit of the balloon with the tissue. In some embodiments, the balloon is inflated to slightly distend tissue and the balloon distending the tissue imaged, in order to establish limits of tissue distension. The balloon can be inflated with any suitable fluid as described herein.

At a step 1840, one or more bleeding locations are identified. The bleeding locations can be identified by a user viewing a screen of a user interface. In some embodiments, the one or more bleeding locations are identified laser pointing with the probe inserted into the patient. Alternatively or in combination, the bleeding tissue locations can be identified with an artificial intelligence algorithm, such as a machine vision algorithm, for example a convolutional neural network.

At a step 1842, the balloon is deflated. The balloon can be deflated slightly to allow fluid to flow around the balloon, for example deflated by an amount within a range from 10% to 30% of the amount of inflation prior to deflation.

At a step 1844, fluid flow around the balloon is activated. The fluid flow can be activated in many ways, for example with an input to a control. The fluid flow may comprise any suitable flow as described herein. In some embodiments, the fluid flow comprises flow from one or more irrigation ports proximal to the balloon and one or more evacuation ports, e.g. irrigation ports, distal to the balloon, so as to establish fluid flow in a proximal to distal direction. While any suitable flow rate can be used, in some embodiments the flow rate is within a range from about 5 milliliters (“ml”) per minute to about 200 ml/minute for example from 10 ml/minute to 100 ml/minute. In some embodiments the fluid flow comprises laminar flow around the balloon.

At a step 1846, the imaged field of view is visualized. The visual field can be visualized in many ways, for example with a user such as a physician viewing a display. Alternatively or in combination, the imaged field of view can be input into an AI algorithm and the field of view visualized with the AI algorithm.

At a step 1848, a static clot and flowing blood are differentiated from the blood in the image. In some embodiments, the static clot is distinguished from flowing blood by movement of the blood in the image. In some embodiments, an origin of flowing blood is identified.

At a step 1850, the one or more bleeding locations is input to the processor and received by the processor.

At a step 1860, a balloon is inflated. In some embodiments, the balloon is re-inflated to substantially the same size as in step 1530, for example to within 15% of the size. In some embodiments, the balloon is inflated slowly while confirming and recording the location of bleeding.

At a step 1870, tissue is treated with energy to decrease bleeding at the one or more locations. The energy may comprise any suitable energy as described herein, such as one or more of thermal energy, light energy, or electrical energy. In some embodiments, the energy is delivered through the balloon, for example light energy delivered through the balloon. In some embodiments, registration of the received locations to the probe is maintained, in order to facilitate alignment of the probe with the received locations.

At a step 1880, the balloon is deflated.

At a step 1885, one or more of steps 1530 to 1580 is repeated.

At a step 1890, the probe is removed from the patient.

Although FIG. 18 shows a method of treating a patient to decrease bleeding in accordance with some embodiments, a person of ordinary skill in the art will recognize many adaptions and variations. For example, some of the steps can be omitted, some of the steps repeated, and the steps can be performed in any order. Also, some of the steps can be performed with the processor as described herein and some of the steps performed manually, and any suitable combination thereof.

### Experimental

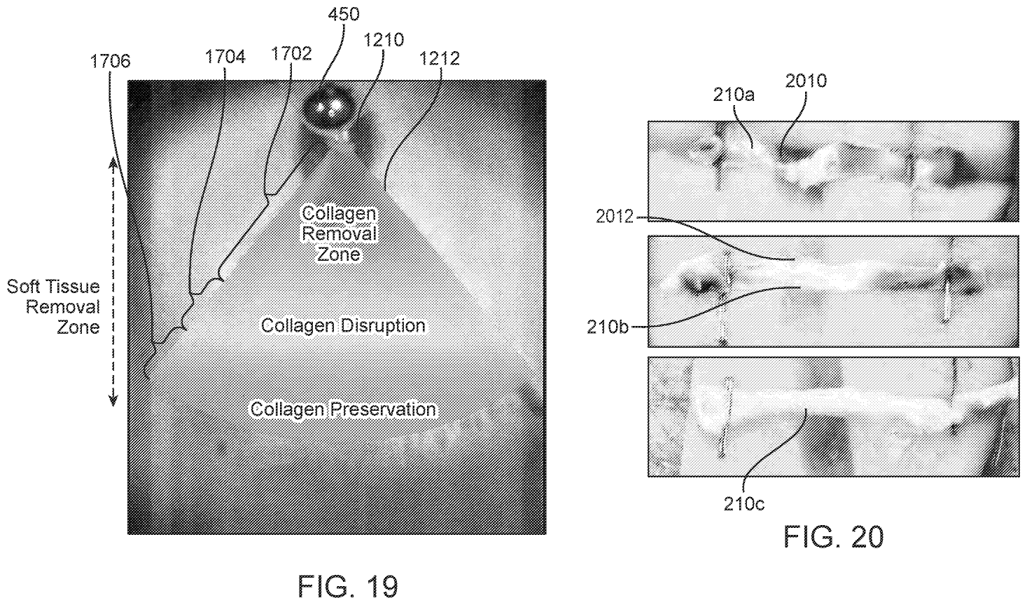

FIG. 19 shows a probe and selective tissue resection zones in accordance with an experiment conducted by the present inventors. The probe 450 comprises a nozzle 1210 configured to release a water jet 1212 as described herein. The water jet 1212 comprises a collagen removal zone 1702, a collagen disruption zone 1704 and a collagen preservation zone 1706. The probe was directed to blood vessels to determine the extent of resection of the blood vessels.

FIG. 20 shows resection of blood vessels 210 and water jet intensities corresponding to a collagen removal zone, a collagen disruption zone and a collagen preservation zone. The blood vessel comprises a substantial amount of connective collagen tissue and can be used as a model for other tissues. For the blood vessel 210a placed at the water jet location corresponding to the collage removal zone, the blood vessel was resected (top) at location 2010. For the blood vessel 210b placed at the collagen disruption zone, the blood vessel was disrupted and show some fraying near the edges of the blood vessel (middle) at location 2012. For the blood vessel 210c treated at the distance corresponding to the collagen removal zone the blood vessel remained intact (bottom).

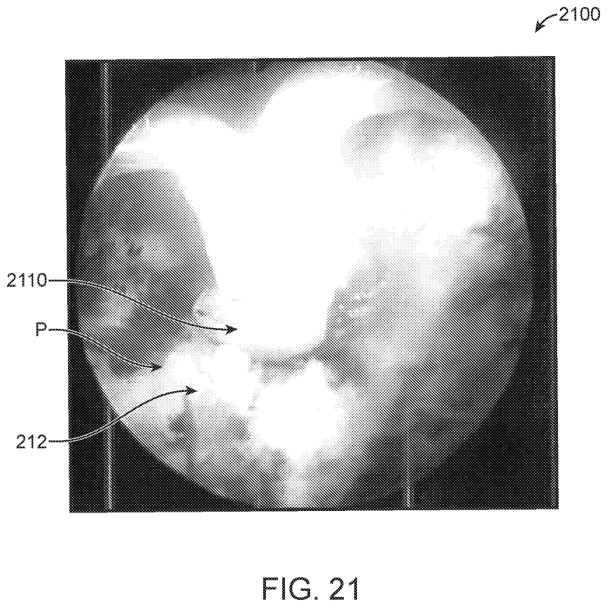

FIG. 21 shows an endoscopic image 2100 of a resected human prostate P, in accordance with some embodiments. The image 2100 shows resected prostate tissue P, collagen filaments 212, and an electrocautery button 2110. The resected tissue shows collagen filaments 212 that appear white and fluffy. The filaments 212 comprise irregular structure and extend a from the tissue bed beneath the filaments. The electrocautery button 2110 comprises a dimeter of about 5 mm across. The collagenous filaments 212 comprise structure with dimensions within a range from about 1 mm to about 10 mm. In some embodiments, the filaments 212 comprise an unstretched length within a range from about 1 mm to about 10 mm and in some embodiments with in a range from about 1 mm to about 5 mm. The unstretched length comprises a distance extending from the boundary of the unresected soft tissue to the end of the filaments. Although the electrocautery button shown comprises a manual electrocautery button, the button could be configured to move in response to processor instructions as described herein.

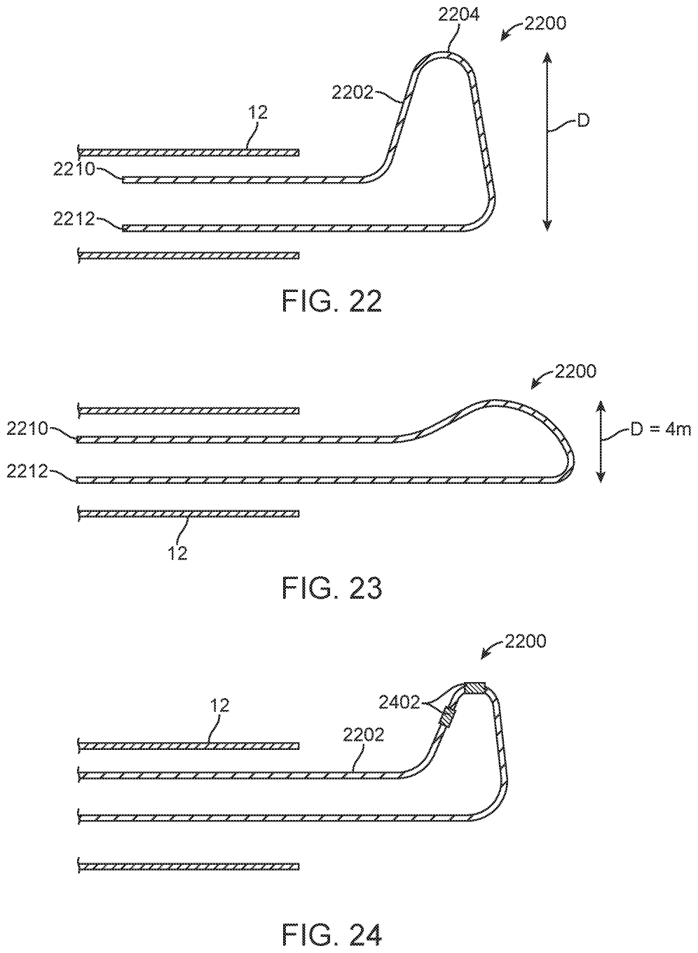

With reference to FIGS. 22, 23 and 24, additional methods and apparatus are illustrated, which are configured to control the position of the energy source precisely and accurately in relation to the tissue surface being treated, in which the energy source comprises a loop structure 2200. In some embodiments, an adjustable member 2202, such as a wire formed of suitable materials, such as nitinol or spring steel, may be shape set or bent to a known geometry forms the loop structure 2200. When both ends or a single end of the loop 2200 is retracted into the shaft a known distance, the loop will reduce its size to a lesser known diameter. When both ends or a single end of the loop is advanced beyond the shaft a known distance, the loop 2200 will increase its size to a larger known diameter D, such as 4 mm. Both ends or a single end of the loop may be controlled using a motor with an encoder (e.g. linear encoder) in line to determine the position of the loop 2200, such that the diameter and position of the loop is always known using geometric calculations or calibrated assembly methods that are stored in the memory of a processor.

In some embodiments, the adjustable member may have a first end 2210 and a send end 2212. In some embodiments, one of the first end 2210 or the second end 2212 is fixed and the other is moveable relative to the shaft. In some embodiments, both the first end 2210 and the second end 2212 are moveable relative to the shaft in order to shape the loop structure 2200.

In some embodiments, the wire may have one, two, three, or more shape set bends 2204. As one end of the wire is manipulated (e.g., pushed, pulled, rotated, or a combination) the wire may take on alternative shapes. In some instances, a loop may be sized by manipulating one or both ends of the wire. The wire may be used for numerous purposes, such as, but not limited to, capturing a tissue structure, delivering energy, guiding an energy delivery device, or some other purpose.

FIG. 24 illustrates a wire 2202 that can be guided to a treatment site. In some cases, the wire may carry one or more electrodes 2402. The electrodes may be activated provide targeted hemostasis. In some cases, multiple electrodes may be carried by the wire and used in combination for bipolar electro cautery and coagulation to a localized treatment site. Two or more electrodes may cooperate, with one driving conducting electrode and another ionically conductive phase. As an electric field is applied across the ionic phase, faradaic reactions occur at the ends of the bipolar electrode even though there may be no direction electrical connection between it and an external power supply. The bipolar electrodes may be used for targeted cautery, coagulation, or some other purpose to reduce heat injury to healthy tissue.

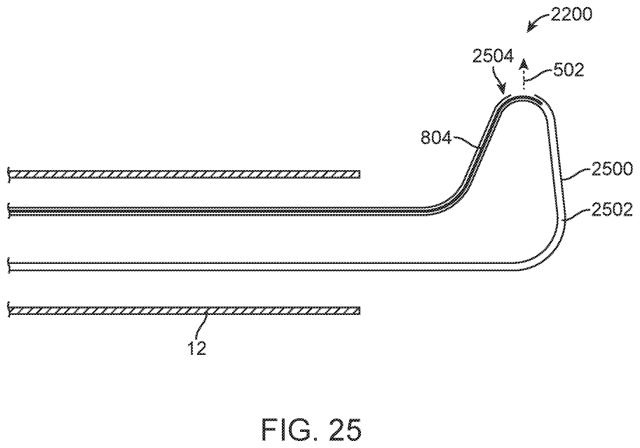

FIG. 25 illustrates a guiding hollow wire 2200. The guiding hollow wire 2500 may be an adjustable tubing loop 2200, which may be adjusted as described herein, such as by pushing, pulling, rotating, or a combination of manipulations to one or two ends of the guiding hollow wire. The guiding hollow wire may be used as described above and may be shape set. The hollow guiding wire 2500 may be formed of any suitable material, and in some embodiments, is formed of Nitinol or spring steel. The hollow guiding wire may have a lumen 2502 therein that guides a fiber 804, such as an optical fiber. The hollow guiding wire can be positioned and manipulated to locate the guiding wire at a target site, and the optical fiber can be fed through the hollow guiding wire to the target site. The hollow guiding wire may have one or more windows or apertures 2504 located within a wall of the hollow guiding wire to allow the optical fiber to emit energy through the window or aperture, such as in the form of a laser light beam 502. In some cases, the optical fiber may be a laser which can be a side firing laser or an end firing laser. The hollow guide wire and window can be formed to deliver the optical fiber to the target location and in the proper orientation to deliver light energy to the target site. The energy source may traverse through the guiding hollow wire and deliver energy to the tissue from a distance controlled by the geometry of the adjustable member. For example, a laser fiber may traverse through a shape set nitinol tube, such that the geometry of the nitinol tube can control the fiber distance to the tissue to deliver the optimized energy required to achieve hemostasis. If the laser fiber is within too close of a proximity to the tissue, there is potential for adjacent tissue damage, and if the laser fiber is too far from the tissue surface, there is potential that insufficient energy is delivered to achieve hemostasis. By controlling the shape of the hollow guide tube, the optical fiber can be located to a degree of certainty sufficient to provide effective optical energy treatment to the target tissue.

In some embodiments, one or more ends of the wire can be coupled to a force sensor which can be used to sense the tissue contacting force of the wire as it is manipulated. For example, the force sensor may detect when a portion of the wire contacts tissue and the wire can be manipulated to either contact tissue or withdraw from tissue contact so the proper energy source and intensity can be delivered to the tissue site. In some instances, delivering RF energy in free can create arcing, and the force sensor can determine when the wire is in contact with tissue to reduce the chance of RF energy causing an arc in free space.

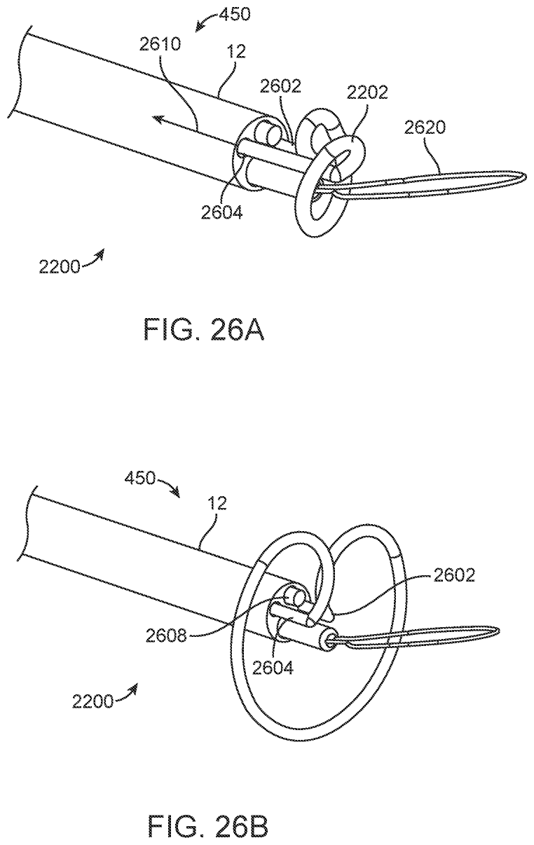

FIGS. 26A and 26B illustrate an active adjustable loop 2200 configured to precisely and accurately control the position of the energy source in relation to the tissue surface being treated. The active adjustable loop may be formed as a helical structure. In some embodiments, an adjustable member 2202, such as a wire which can be formed from suitable materials such as nitinol or spring steel, may be shape set or bent to a known geometry. A first end 2602 of the helix may remain fixed in the shaft 12 of the probe 450. A second end 2604 of the helix may be allowed to translate axially. The second end of the helix may translate in a direction along the elongate axis 2610 of the probe. When the second end of the helix is translated proximally (in relation to the anatomy), the helix can be caused to increase proportionately in size to a known geometry and diameter (see FIG. 26B). As the second end of the helix is translated distally (in relation to the anatomy), the helix can be caused to decrease proportionately in size to a known geometry and diameter (see FIG. 26A). In some cases, either the first end, the second end, or both may be rotated to change the geometry of the helix, such as by expanding the helix to a desired diameter. The second end of the helix may be controlled using a motor with an encoder (e.g. linear encoder) in line to determine the position of the loop, such that the diameter and position of the loop is always known using geometric calculations or calibrated assembly methods that are stored in the memory of a processor. A standard surgical snare 2620 may be provided to capture and retain a tissue structure, which in some cases may be a medial lobe of a prostate.

In some embodiments to control the position of the energy source precisely and accurately to the tissue surface, the adjustable member may be attached to a force sensing element or transducer. Force applied to the adjustable member would be measurable and similarly, the force of the adjustable member against tissue may also be measured.

In some embodiments, the adjustable member may be electrically conductive. In some cases, RF energy may be passed through the adjustable member and conducted through the tissue via a grounding pad (e.g. monopolar energy). In some embodiments, there may be an insulating material between two portions of the adjustable member at the distal end where energy is applied to the tissue. Both portions of the wire may be electrically isolated from each other, such that RF energy may be passed between both portions of the wire (e.g. bipolar energy).

In some embodiments to control the position of the energy source precisely and accurately to the tissue surface, the probe 450 may include an imaging device, such as an endoscope for imaging and observing the tissue at loop 2200.

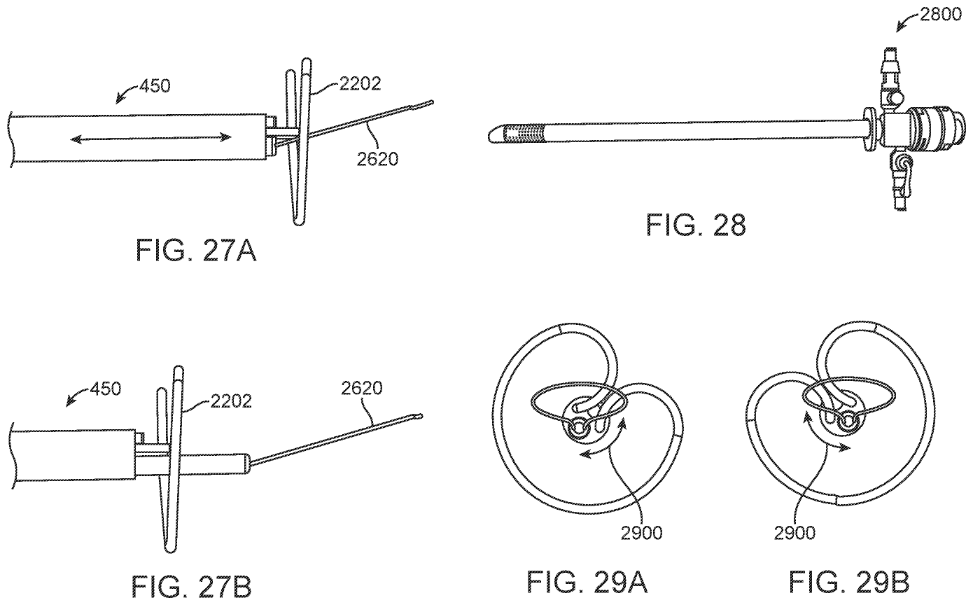

With reference to FIGS. 27A and 27B, in some embodiments, the adjustable member 2202 may be activated with energy and the probe can translate axially to treat tissue within the prostatic cavity. In some cases, a snare 2620 extending from the probe may remain fixed in space as the probe translates along the elongate axis 1250 of the probe. For example, the snare may be manipulated to capture a tissue structure, such as the medial lobe of the prostate, and the snare may then be free to slide within the probe. In other words, once the snare captures the target tissue, the snare may remain stationary while the probe translates axially.

FIG. 28 shows a resectoscope sheath 2800 usable with a probe, in accordance with some embodiments.

With reference to FIGS. 29A and 29B, the probe may be configured to rotate about its longitudinal axis. This motion 2900 may also rotate the active loop to present different geometries to different portions of the tissue to be treated, such as to coagulate tissue or blood in tighter or smaller locations, such as in corners.

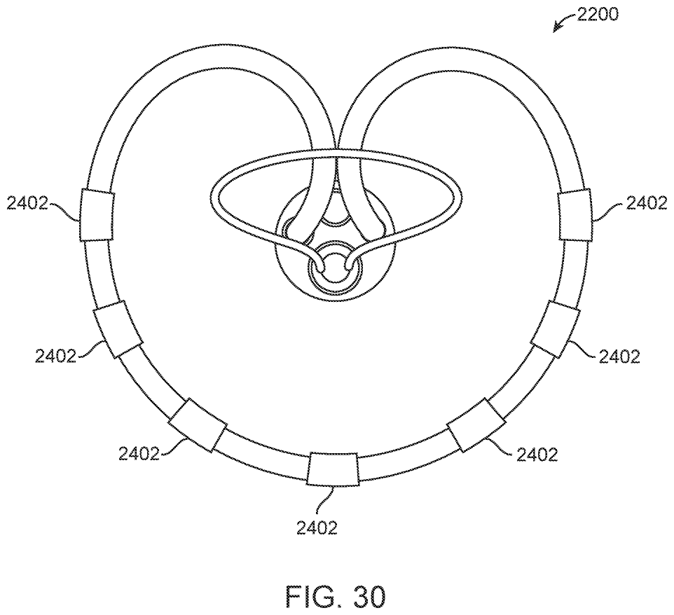

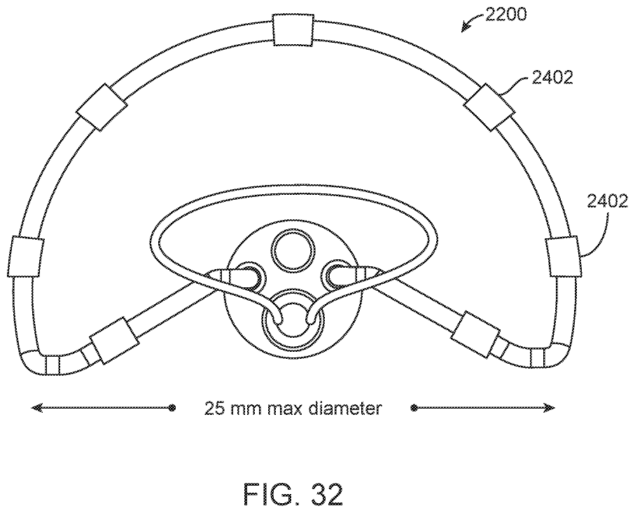

FIGS. 30 and 32 illustrate an active adjustable loop 2200 carrying one or more electrodes 2402. The active adjustable loops 2200 may take any suitable shape or size and may be configured to be adjustable in their size and/or shape, such as by manipulating one or more ends of the loop. For example, energy may be coupled or delivered through the adjustable member, and a single or multiple electrodes, or electrically conductive members, may be attached to the adjustable member. Electrically conductive wiring may be connected from the electrodes through the probe shaft and connected to an energy generator source. Energy may pass from the generator source through the wiring to the single or multiple electrodes on the adjustable member. In some embodiments, RF energy is used to treat the tissue. RF energy may be passed between the electrodes present on the adjustable member (e.g. bipolar energy) or to a grounding pad on the patient (e.g. monopolar energy).

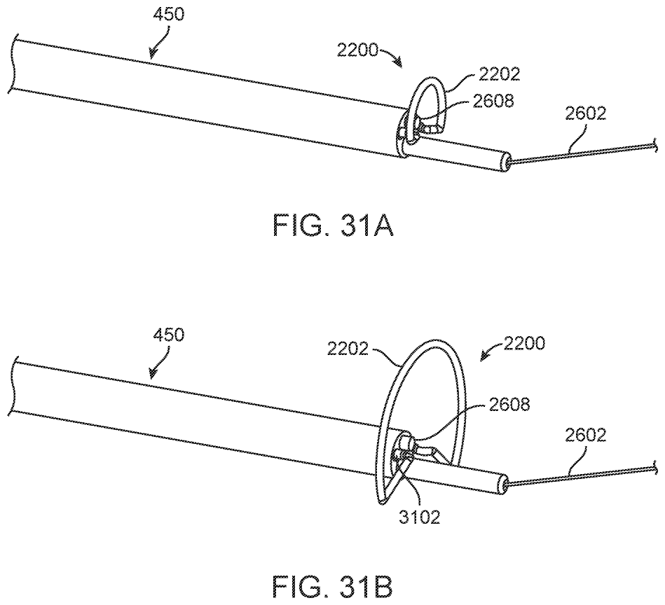

With reference to FIGS. 31A and 31B an adjustable member 2202 may be comprised of a set bend material and may be formed with a predetermined geometry, such as one or more bends. A loop 2200 of the adjustable member may extend distally to the probe 450 and be biased in a direction relative to the probe. As the loop is extended further beyond the probe, the loop may expand in size and return to a predefined orientation and size, such as expanding in diameter. Thus, the size and shape of the adjustable member may be controlled by advancing or retracting the loop, by manipulating a first or second wire that forms the loop. The adjustable member may carry one or more electrodes, as has been described herein. In some embodiments, the adjustable member is biased to allow an imaging system 2608 to have a clear line of sight. In some cases, a guard 3102 is used to reduce the tendency of the adjustable member from obstructing the view of the imaging system.

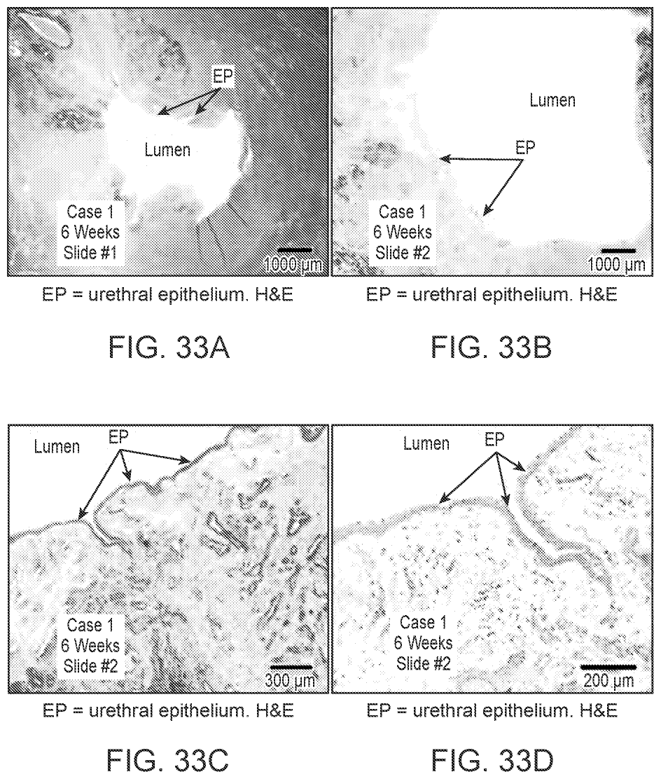

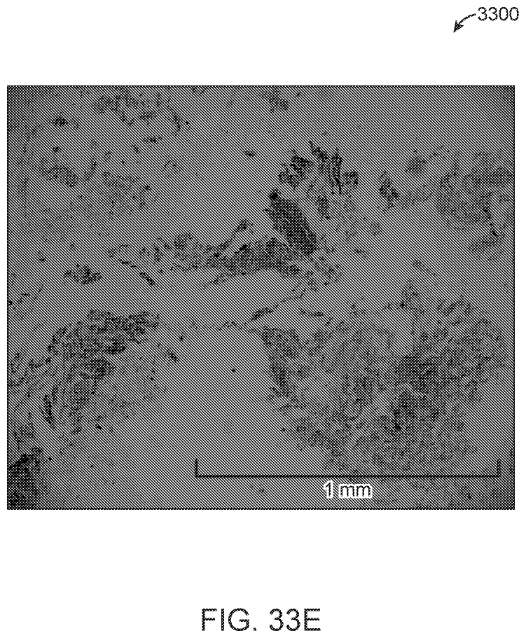

FIGS. 33A, 33B, 33C, and 33D show a canine prostate tissue sections about six weeks after treatment with a water jet, in which the tissue has healed to substantially decrease the collagenous fibers. As can be seen from these images of tissue sections that were obtained with H&E staining, an epithelial layer (EP) has grown over the resection bed to define a urethral lumen.

FIG. 33A shows a first slide from case 1, approximately 6 weeks post-op with a magnification scale of 1000 um.

FIG. 33B shows a second slide from case 1, approximately 6 weeks post-op with a magnification scale of 1000 um.

FIG. 33C shows an image from slide 2 with a magnification scale of approximately 300 um.

FIG. 33D shows an image from slide 2 with a magnification scale of approximately 200 um.

FIG. 33E shows a histological tissue section 3300 of a collagenous tissue bundle of fibers as described herein. This study was conducted to understand the type of tissue remaining after water jet based aqua ablation as described herein. A specimen was collected from a fresh frozen cadaver which prostate was accessed and treated using the aqua ablation system with all tissues ablated by high velocity waterjet where the surgical plan was limited to not penetrate the prostatic capsule. The sample shown in FIG. 33E was taken from the attached, free floating white fiber found closest to the jet origin. Formalin fixed samples were trimmed into tissue processing cassettes, routinely processed for paraffin histology. Paraffin blocks were sectioned on a microtome at 4-6 microns, mounted on glass slides and stained with Hematoxylin & Eosin (H&E) for light microscopic evaluation by the study pathologist. Microscopic analysis for histology revealed the remaining tissue (fluffies) comprise tissue consistent with collagen fragments. As can be seen in the slide, there are regions with tissue and regions without tissue, which is consistent with the tissue comprising collagen filaments, e.g. fluffies, as described herein.

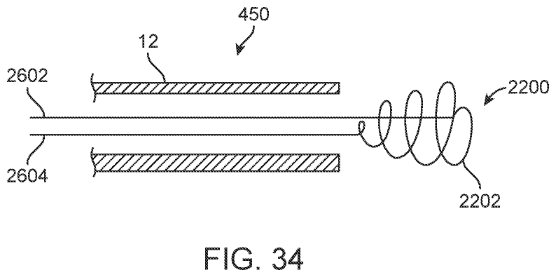

FIG. 34 shows geometric control of an energy loop, in accordance with some embodiments. The adjustable energy loop 2200 may be formed as a helical structure. In some embodiments, an adjustable member 2202, such as a wire which can be formed from suitable materials such as nitinol or spring steel, may be shape set or bent to a known geometry. A first end 2602 of the helix may be attached to a clock spring on the proximal end of the shaft 12 of the probe 450. The first end may also or instead, attached to a rack to provide stiffness and reduce buckling loads in the shaft. The rack can be driven using a rack and pinion system and an actuator. The second end of the helix may translate in a direction along the elongate axis of the probe. A second end 2604 of the helix may be fixed. When the first end of the helix is translated proximally (in relation to the anatomy), the helix can be caused to increase proportionately in size to a known geometry and diameter. As the first end of the helix is translated distally (in relation to the anatomy), the helix can be caused to decrease proportionately in size to a known geometry and diameter. The first end of the helix may be controlled using a motor with an encoder (e.g. linear encoder) in line to determine the position of the loop, such that the diameter and position of the loop is always known using geometric calculations or calibrated assembly methods that are stored in the memory of a processor. When the first end is extended so that the loop begins at location 3, the diameter of the loop may be 10 mm. When the first end is extended so that the loop begins at location 4, the diameter of the loop may be 17.5 mm. When the first end is extended so that the loop begins at location 5, the diameter of the loop may be 25 mm.

By controlling the loop pitch, diameter, material, and length, a spring constant may be defined. Based on the spring constant, a desired or optimal pressure may be applied to the tissue so as not to injure the tissue. As discussed herein, a force measuring device may be placed on one or both legs to measure the force applied to the leg and calculate a pressure applied to the tissue by the loop.

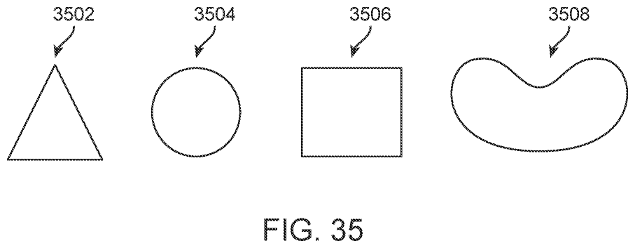

FIG. 35 shows cross sectional geometries that may be configured to target anatomy with a structure such as a loop 2200 as described herein, such as a square 3502, circle 3504, triangle 3506, and other shapes. A bean shape 3508 may be the expected geometry following resection.

As described herein, the computing apparatuses and systems described and/or illustrated herein broadly represent any type or form of computing apparatus or system capable of executing computer-readable instructions, such as those contained within the modules described herein. In their most basic configuration, these computing apparatus(s) may each comprise at least one memory apparatus and at least one physical processor.

The term “memory” or “memory apparatus,” as used herein, generally represents any type or form of volatile or non-volatile storage apparatus or medium capable of storing data and/or computer-readable instructions. In one example, a memory apparatus may store, load, and/or maintain one or more of the modules described herein. Examples of memory apparatus comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory apparatus. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing apparatus, may cause the computing apparatus to perform one or more tasks, such as the method step.

In addition, one or more of the apparatus described herein may transform data, physical apparatus, and/or representations of physical apparatus from one form to another. Additionally or alternatively, one or more of the modules recited herein may transform a processor, volatile memory, non-volatile memory, and/or any other portion of a physical computing apparatus from one form of computing apparatus to another form of computing apparatus by executing on the computing apparatus, storing data on the computing apparatus, and/or otherwise interacting with the computing apparatus.

The term “computer-readable medium,” as used herein, generally refers to any form of apparatus, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

As used herein, light refers to electromagnetic energy such as one or more infrared electromagnetic radiation, near infrared electromagnetic radiation, visible electromagnetic radiation, or ultraviolet electromagnetic radiation.

The present disclosure includes the following numbered clauses.

Clause 1. A probe for treating tissue comprising: an elongate shaft; an expandable balloon coupled to the elongate shaft; a light source to emit light through the balloon; and an endoscope viewing port, the endoscope viewing port configured to view tissue through the balloon.

Clause 2. The probe of clause 1, wherein the light source comprises a plurality of light sources.

Clause 3. The probe of clause 1, wherein the endoscope viewing port is located within the balloon.

Clause 4. The probe of clause 1, wherein the endoscope viewing port is located outside the balloon and configured to view the tissue through a first portion of the balloon and a second portion of the balloon.

Clause 5. The probe of clause 1, wherein the balloon comprises an optically transmissive material configured to allow the endoscope to image tissue through the balloon.

Clause 6. The probe of clause 1, wherein the balloon comprises a transparent material.

Clause 7. The probe of clause 1, wherein the balloon comprises a substantially transparent material configured to transmit light at red light, blue light, and green light and the endoscope is configured to generate a color image of the tissue through the balloon.

Clause 8. The probe of clause 1, wherein the balloon comprises sufficient transparency to view the tissue through the balloon with a resolving power of 100 through the endoscope.

Clause 9. The probe of clause 8, wherein the balloon material is sufficiently transparent to enable a visualization system to visualize blood flow, fluffy fibers, and anatomical structure to a resolving power of 100 μm.

Clause 10. The probe of clause 8, wherein the balloon comprises one or more of an elastomer, silicone, rubber, a thermoplastic rubber elastomer (e.g. ChronoPrene™), latex, polyethylene terephthalate (“PET”), urethane, polyurethane, polytetrafluoroethylene (“PTFE”), a conformal coating, a poly(p-xylylene) polymer, a chemically deposited poly(p-xylylene) polymer, Parylene™, nylon, poly(ether-b-amide), plasticizer-free poly(ether-b-amide), Pebax®, nylon elastomer.

Clause 11. The probe of clause 10, further comprising a coating on one or more of an inside or an outside of the balloon and optionally wherein the coating comprises one or more of starch, silicon, silica or corn starch.

Clause 12. The probe of clause 8, wherein the balloon comprises sufficient transparency to view the tissue through a first portion of the balloon and a second portion of the balloon with the resolving power of 100 μm, the first portion located near the tissue, the second portion located near the endoscope and closer to the endoscope than the first portion.

Clause 13. The probe of clause 1, wherein the balloon comprises a material with a layer configured to absorb at least about 50% of the light transmitted from the light source at a first wavelength to heat tissue with the balloon and to transmit at least about 50% of light from the tissue toward the endoscope at a second wavelength to image the tissue with the second wavelength and optionally wherein the probe is configured to inflate the balloon with a liquid to conduct heat from balloon.

Clause 14. The probe of clause 1, wherein the endoscope comprises a polychromatic light source to illuminate the tissue and generate a color image of the tissue.

Clause 15. The probe of clause 1, wherein the light source comprises an optical fiber extending toward a distal tip to emit light energy.

Clause 16. The probe of clause 1, wherein the light source comprises a laser diode located on the shaft to emit light energy.

Clause 17. The probe of clause 1, wherein the balloon is configured to expand from a narrow profile configuration for insertion into a tissue space to an expanded profile to contact tissue.

Clause 18. The probe of clause 17, wherein the balloon in the narrow profile configuration comprises one or more of an approximately cylindrical shape within 25% of the probe diameter, a balloon comprising a diameter larger than a shaft of the probe and a tapered shape profile near a distal end of the balloon to facilitate advancement of the probe, or a balloon wrapped around the shaft to decrease a cross-sectional size of the balloon.

Clause 19. The probe of clause 1, wherein the light source is configured to translate and rotate in relation to the balloon and the elongate shaft to coagulate tissue through the balloon.

Clause 20. The probe of clause 1, shaft comprises a fluid flushing lumen extending to a flushing opening on a first side of the balloon and an evacuation lumen extending to an evacuation opening on the second side of the balloon, in order to establish fluid flow around the balloon to remove material between the tissue and the balloon.

Clause 21. The probe of clause 20, wherein the light source is configured to emit light toward the tissue along an optical path at an oblique angle to an elongate axis of the shaft and wherein the angle is within a range from about 15 degrees to about 85 degrees.

Clause 22. The probe of clause 21, wherein the optical path extends in a direction corresponding to a direction of fluid flow around the balloon to decrease obscuration of the beam directed to tissue.

Clause 23. The probe of clause 21, wherein the optical path extends in a direction opposite a direction of fluid flow around the balloon.

Clause 24. The probe of clause 1, wherein the shaft comprises a lumen coupled to the balloon to inflate the balloon.

Clause 25. The probe of clause 1, wherein the balloon comprises a first layer and a second layer configured to separate from the first layer to form a channel with a liquid between the first layer and the second layer.

Clause 26. The probe of clause 25, further comprising a lumen extending along the shaft to provide a liquid to channel and separate the first layer from the second layer.

Clause 27. The probe of clause 25, wherein the probe comprises a first lumen to provide fluid to an interior of the balloon inside the first layer and a second lumen to provide a liquid to the channel.

Clause 28. The probe of clause 27 and wherein the fluid comprises a gas.

Clause 29. The probe of clause 27 and wherein the liquid comprises a chromophore.

Clause 30. The probe of clause 1, wherein the shaft is coupled to a handpiece to move the light source.

Clause 31. The probe of clause 1, wherein the shaft is configured to couple to a linkage operatively coupled to a processor to move the light source with the linkage in response to instructions from the processor.

Clause 32. The probe of clause 1, wherein the energy source comprises an energy source to heat tissue to decrease bleeding, the energy source comprising one or more of a thermal energy source, a cooling energy source, a light beam, an electrode, a radiofrequency (RF) electrode, a monopolar electrode, a bipolar electrode, a loop electrode, a button electrode, ultrasound, high intensity focused ultrasound, ultrasonic cavitations, a plasma energy source, a microwave energy source, or a cryogenic energy source.

Clause 33. A probe for treating tissue, the probe comprising: a shaft; a balloon coupled to the shaft, the balloon configured to expand to a radius; a light source located within the balloon, wherein the light source is configured to provide an irradiance profile to the balloon when the balloon has expanded to the radius.

Clause 34. The probe of clause 33, wherein the irradiance profile comprises a predetermine irradiance profile over an area when the balloon has expanded to a predetermined radius at a treatment location.

Clause 35. A probe to treat tissue, comprising: an elongate shaft comprising a lumen extending to a distal end; an optical fiber within the lumen, the optical fiber configured to extend beyond the distal end of the lumen and deflect toward the tissue; and an engagement structure coupled to the end of the optical fiber, the engagement structure comprising an engagement surface to contact the tissue, the engagement surface comprising an area larger than a cross-section of the optical fiber to decrease pressure to the tissue.

Clause 36. The probe of clause 35, wherein the engagement surface comprises a dimension across within a range from 2 to 10 mm and optionally within a range from 3 to 7 mm.

Clause 37. The probe of clause 35, wherein the engagement surface comprises one or more of a curved surface, a flat surface, an inclined surface or a bevel to allow the engagement structure to slide along a resected tissue with filaments.

Clause 38. The probe of clause 35, wherein engagement structure comprises one or more of a ball, a cylinder or a roller.

Clause 39. The probe of clause 35, wherein the optical fiber extends into the engagement structure and wherein the engagement structure comprises one or more of an opening or an optically transmissive material to transmit light energy from a distal tip of the optical fiber to the tissue.

Clause 40. The probe of clause 35, further comprising a sheath over the optical fiber, the sheath configured to deflect the optical fiber, wherein the sheath is dimensioned to extend from the end of the lumen toward the tissue.

Clause 41. The probe of clause 40, wherein the sheath comprises torsional stiffness to rotate the engagement structure in relation to lumen when the engagement structure contacts the tissue.

Clause 42. The probe of clause 41, wherein the sheath is configured to contact the tissue with a first amount of force and wherein the torsional stiffness is sufficient to rotate the engagement structure and overcome frictional force related to the first amount of force when the engagement structure contacts the tissue.

Clause 43. The probe of clause 35, wherein the tissue engagement structure is coupled to a handpiece to move the tissue engagement structure.

Clause 44. The probe of clause 35, wherein the tissue engagement structure is coupled to a linkage operatively coupled to a processor to move the tissue engagement with the linkage in response to instructions from the processor.

Clause 45. A probe for treating tissue, the probe comprising: a shaft configured to move with one or more of rotational or translational movement; and a light source coupled to the shaft, the light source configured to emit an elongate beam with an elongate cross-section, the shaft configured to scan the beam in a direction transverse to the elongate beam.

Clause 46. The probe of clause 45, wherein the elongate cross-section comprises a longest dimension across and a shortest dimension across, the light source configured to scan the beam in the direction transverse to longest dimension across.

Clause 47. The probe of clause 45, wherein the shaft is configured to rotate to scan the beam in the direction transverse to the longest dimension across.

Clause 48. The probe of clause 45, wherein the probe comprises an elongate axis and wherein an axis of the elongate distance across is within 15 degrees of parallel to the elongate axis of the probe.

Clause 49. The probe of clause 45, wherein the light source comprises an optical fiber with a tapered end portion to emit the elongate beam.

Clause 50. The probe of clause 45, wherein the light source comprises an optical fiber with a tapered end with a reflective surface to emit the elongate beam in a direction and orientation with respect to the optical fiber to focus the elongate beam toward the tissue and optionally wherein the direction and orientation comprise a predetermined direction and orientation.

Clause 51. The probe of clause 45, wherein the light source comprises a plurality of optical fibers arranged in an array to emit the elongate beam.

Clause 52. The probe of clause 51, further comprising an array of lenses coupled to ends of the plurality of optical fibers to emit the elongate beam.

Clause 53. The probe of clause 45, wherein the light source comprises an optical fiber coupled to a lens to generate the elongate beam.

Clause 54. The probe of clause 45, wherein the light source comprises an optical fiber extending along the probe and wherein the optical fiber comprises a bend relative to an elongate axis of the probe in order to direct a light beam to the tissue at a non-parallel angle to the elongate axis of the probe.

Clause 55. The probe of clause 54, wherein the optical fiber comprises a bend of 90 degrees to direct the beam at angle of approximately 90 degrees to the elongate axis of the probe.

Clause 56. The probe of clause 45, wherein the elongate beam comprises a light sheet.

Clause 57. The probe of clause 45, wherein the elongate cross-section comprises an annular cross-section extending radially outward from the probe.

Clause 58. The probe of clause 57, wherein the light source comprises an optical fiber coupled to a conical mirror.

Clause 59. The probe of clause 57, wherein the beam comprises a conical beam extending from a conical mirror.

Clause 60. The probe of clause 57, wherein the probe comprises an axis and the probe are configured to translate along the axis to scan the beam transverse to the elongate beam.

Clause 61. A probe to treat tissue, comprising: a shaft; a nozzle coupled to the shaft, the nozzle located at a first location on the shaft, the nozzle configured to release a water jet toward the tissue; a light source coupled to the shaft, the light source configured to direct a light beam to the tissue from a second location of the shaft, the second location different from the first location.

Clause 62. The probe of clause 61, wherein the nozzle is aligned relative to an elongate axis of the shaft to direct the water jet to a first region of tissue and wherein the light source is aligned relative to the axis to direct the light beam to a second region of tissue different from the first region when the nozzle is directed toward the first region.

Clause 63. The probe of clause 62, wherein the first region does not overlap with the second region.

Clause 64. The probe of clause 62, wherein the probe is configured to rotate about the elongate axis of the probe and to translate along the elongate axis and wherein the first location and the second location are located along the probe at spaced apart locations and a similar rotational angle with respect to the elongate axis and wherein the probe is configured to translate the light source along the elongate axis to treat the region of tissue treated with the water jet.

Clause 65. The probe of clause 61, wherein the nozzle is aligned relative to an elongate axis of the shaft to direct the water jet to a first region of tissue and wherein the light source is aligned relative to the elongate axis to direct the light beam to a second region of tissue overlapping with the first region when the nozzle is directed toward the first region.

Clause 66. The probe of clause 61, wherein the shaft comprises a first side and a second side and wherein first side comprises the first location and the second location.

Clause 67. The probe of clause 61, wherein the first location is on a first side of the shaft and the second location is on a second side of the shaft with a midline between the first side and the second side.

Clause 68. The probe of clause 61, wherein the light source comprises an optical fiber coupled to an output aperture at the second location.

Clause 69. The probe of clause 61, wherein the light source comprises one or more of an optical fiber, a bent optical fiber, a prism, a lens or a mirror.

Clause 70. The probe of clause 61, further comprising a high-pressure lumen coupled to the nozzle and wherein an optical fiber extends along shaft inside the high-pressure lumen.

Clause 71. The probe of clause 70, wherein the nozzle is coupled to the high-pressure lumen at the first location and the optical fiber extends to an output aperture at the second location.

Clause 72. The probe of clause 61, further comprising a high-pressure lumen coupled to the nozzle and an optical fiber extending to an end located outside the high-pressure lumen.

Clause 73. The probe of clause 61, wherein the light source comprises an optical fiber in a sheath extending along the shaft and wherein the nozzle is fluidically coupled to a lumen of a high pressure tube, the high pressure tube adjacent the sheath.

Clause 74. The probe of clause 73, wherein the shaft comprises a tube and the sheath and the high-pressure tube extend along an interior of the tube.

Clause 75. The probe of clause 61, wherein the shaft comprises an axis and the nozzle comprises an internal channel to direct the water jet at a first angle to the axis and wherein the light source is configured to emit the light beam at a second angle to the axis, the first angle different from the second angle.

Clause 76. The probe of clause 75, wherein the second angle comprises an oblique angle within a range from about 20 degrees to about 70 degrees.

Clause 77. The probe of clause 75, wherein the first angle is within a range from about 75 degrees to about 105 degrees.

Clause 78. An apparatus to treat tissue, the apparatus comprising: a probe comprising an energy source to heat tissue to decrease bleeding; a linkage coupled to the probe; a processor coupled to the linkage to move the probe, wherein the processor is configured with instructions to, receive an input corresponding to a location of bleeding tissue; and direct the energy source to a region of tissue to decrease bleeding in response to the input location.

Clause 79. The apparatus of clause 78, wherein the probe comprises the probe of any one of the preceding clauses.

Clause 80. The apparatus of clause 78, wherein the processor is configured to scan the energy source at distance from the location to decrease bleeding of the tissue at the location.

Clause 81. The apparatus of clause 78, wherein the input comprises an input from a user interface, and wherein the user interface comprises an image of the tissue and the input corresponds to a location of bleeding of the tissue.

Clause 82. The apparatus of clause 78, wherein the probe is configured to emit an aiming beam with an amount of energy visible to a user.

Clause 83. The apparatus of clause 82, wherein the energy source comprises a laser beam and wherein the processor is configured with instructions to increase an amount of energy of the laser beam from a first amount of energy to aim the laser beam to a second amount of energy to coagulate tissue away from the location.

Clause 84. The apparatus of clause 82, wherein aiming beam comprises a first wavelength of light and the energy source comprises a laser beam comprising a second wavelength of light different from the first wavelength of light.

Clause 85. The apparatus of clause 84, wherein the aiming beam comprises a first intensity and the laser beam comprises a second intensity greater than the first intensity.

Clause 86. The apparatus of clause 82, the processor configured with instructions for the user to adjust a position of the aiming beam and wherein the processor is configured with instructions for the user to provide the input when the aiming beam has been aligned with the bleeding location in order to initiate a scan of the energy source away from the location.

Clause 87. The apparatus of clause 78, wherein the processor is configured to scan the energy source around the location a plurality of times.

Clause 88. The apparatus of clause 78, wherein the energy source comprises a light beam and the processor is configured to rotate and translate an optical structure to scan the beam, the optical structure comprising one or more of a lens, a prism, a mirror or a distal end of an optical fiber.

Clause 89. The apparatus of clause 78, wherein the energy source comprises a light beam and the probe comprises a balloon and the processor is configured to receive the input prior to expansion of the balloon to engage tissue and to scan the beam through the balloon after the balloon has expanded to engage the tissue.

Clause 90. The apparatus of clause 78, wherein the energy source comprises one or more of a thermal energy source, a cooling energy source, a light beam, an electrode, a radiofrequency (RF) electrode, a monopolar electrode, a bipolar electrode, a loop electrode, a button electrode, ultrasound, high intensity focused ultrasound, ultrasonic cavitations, a plasma energy source, or a cryogenic energy source.

Clause 91. The apparatus of clause 78, further comprising a Doppler ultrasound image and wherein the processor is configured with instructions to receive an input corresponding to the location of the bleeding tissue in the Doppler ultrasound image.

Clause 92. The apparatus of clause 91, wherein the processor is configured with instructions to identify the location of bleeding tissue in response to a change in a velocity of a fluid from the Doppler ultrasound image and optionally wherein the fluid comprises blood.

Clause 93. The apparatus of clause 92, wherein the change in velocity comprises a decrease in velocity of the fluid along a flow path.

Clause 94. The apparatus of clause 92, wherein the change in fluid velocity corresponds to a pulsatile flow of the fluid.

Clause 95. The apparatus of clause 92, wherein the fluid comprises blood flowing along a blood vessel and wherein the fluid is released through an opening in the vessel wall.

Clause 96. The apparatus of clause 95, wherein the fluid is released into a second fluid, the second fluid comprising a lower velocity than the first fluid and wherein the bleeding location is identified in response to a change in direction of the fluid through the vessel wall.

Clause 97. The apparatus of clause 92, wherein the bleeding location is identified by registering a first image of the tissue prior to tissue resection with a second image of the tissue after tissue resection and wherein the change in velocity of the fluid is identified at least in part based on a change between the first image and the second image and optionally wherein a blood vessel of the first image is measured with a corresponding blood vessel from the second image.

Clause 98. A method of treating tissue to decrease bleeding, the method comprising treating tissue with the apparatus or probe of any one of the preceding clauses.

Clause 99. The method of clause 92 wherein the tissue comprises filaments of collagenous tissue comprising an unstretched length within a range from about 1 mm to about 10 mm extending from a boundary of unresected tissue into an enclosed tissue space.

Clause 100. A method of treating tissue of a patient, the method comprising: inserting a probe into the patient, the probe comprising a nozzle to release a water jet; resecting tissue with a water jet, wherein the resected tissue comprises filaments and one or more ruptured blood vessels; inserting a resectoscope into the patient to treat bleeding from the one or more ruptured blood vessels.

Clause 101. The method of clause 100, wherein the filaments comprise an unstretched length within a range from about 1 mm to about 10 mm extending from a boundary of unresected tissue into an enclosed tissue space.

Clause 102. The method of clause 100, wherein the resectoscope comprises an endoscope comprising light and a lens for viewing the filaments.

Clause 103. The method of clause 100, wherein the resectoscope comprises one or more of an electrode or an optical fiber for cauterizing the one or more ruptured blood vessels.

Clause 104. The method of clause 100, further comprising removing the probe comprising the water jet prior to inserting the resectoscope.

Clause 105. A method of treating a patient, comprising: inserting a probe into a patient, the probe comprising a balloon; inflating the balloon; deflating the balloon; identifying bleeding locations with the balloon deflated; inflating the balloon; treating tissue at the bleeding locations with the balloon inflated.

Clause 106. The method of clause 105, further comprising flushing fluid on a first side of the balloon and evacuating fluid on a second side of the balloon to provide fluid flow around the balloon when the balloon has been deflated.

Clause 107. The method of clause 106, wherein the balloon is deflated by no more than 30% of the volume of the balloon when the balloon has been inflated.

Clause 108. The probe of any one of the preceding clauses, wherein the light source is used to measure the surface temperature of the tissue being treated.

Clause 109. The probe of any one of the preceding clauses, wherein the light source may be used to measure the distance from the light source to the surface of the tissue being treated.

Clause 110. A probe for treating tissue comprising: an elongate shaft; an adjustable wire member housed within the elongate shaft; an energy source coupled to the adjustable wire member; and an endoscope viewing port, the endoscope viewing port configured to view tissue.

Clause 111. The probe of clause 110, wherein the endoscope viewing port is configured to view tissue through a balloon.

Clause 112. The probe of clause 110, wherein the adjustable member comprises of a helical wire with a fixed and adjustable end whose geometry is controlled via axial translation of the second adjustable end of the wire.

Clause 113. The probe of clause 111, wherein the adjustable member is a single electrically conductive member that can deliver electrical energy to the tissue.

Clause 114. The probe of clause 111, wherein the adjustable member is split into two electrically conductive members that can deliver electrical energy to the tissue using the two separate conductive members as the dipoles.

Clause 115. The probe of clause 111, wherein the adjustable member is non-conductive and a single or multiple electrically conductive members are mounted on to the adjustable member and optionally wherein said electrically conductive members are capable of delivering electrical energy to the tissue using said members as dipoles or a grounding pad to said members as the dipoles.

Clause 116. The probe of clause 111, wherein the adjustable member comprises a tubular shaft wherein an energy source is housed within said tubular shaft and optionally wherein said energy source is configured to deliver energy to the tissue using the adjustable tubular shaft to control a position of the energy delivery.

Clause 117. The probe of clause 110, wherein adjustable member comprises of a loop wire with two adjustable ends whose geometry is controlled via simultaneous axial translation of the both adjustable ends of the wire.

Clause 118. The probe of clause 117, wherein the adjustable member is a single electrically conductive member that can deliver electrical energy to the tissue.

Clause 119. The probe of clause 117, wherein the adjustable member is split into two electrically conductive members that can deliver electrical energy to the tissue using the two separate conductive members as the dipoles.

Clause 120. The probe of clause 117, wherein the adjustable member is non-conductive and a single or multiple electrically conductive members are mounted on to the adjustable member and optionally wherein said electrically conductive members are capable of delivering electrical energy to the tissue using said members as dipoles or a grounding pad to said members as the dipoles.

Clause 121. The probe of clause 117, wherein the adjustable member comprises a tubular shaft and wherein an energy source is housed within said tubular shaft and optionally wherein said energy source is configured to deliver energy to the tissue using the adjustable tubular shaft to control a position of the energy delivery.

Clause 122. The probe of clause 110, wherein adjustable member comprises a loop wire with a first fixed end and a second adjustable end whose geometry is controlled via axial translation of the adjustable end of the wire.

Clause 123. The probe of clause 122, wherein the adjustable member comprises a single electrically conductive member that can deliver electrical energy to the tissue.

Clause 124. The probe of clause 122, wherein the adjustable member is split into two electrically conductive members that can deliver electrical energy to the tissue using the two separate conductive members as the dipoles.

Clause 125. The probe of clause 122, wherein the adjustable member is non-conductive and a single or multiple electrically conductive members are mounted on to the adjustable member and optionally wherein said electrically conductive members are capable of delivering electrical energy to the tissue using said members as dipoles or a grounding pad to said members as the dipoles.

Clause 126. The probe of clause 122, wherein the adjustable member is a tubular shaft wherein an energy source is housed within said tubular shaft and optionally wherein said energy source is configured to deliver energy to the tissue using the adjustable tubular shaft to control a position of the energy delivery.

Clause 127. The probe of clause 110, wherein the adjustable member is connected to a force sensor to determine the contact force between said adjustable member and tissue surface.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A probe for treating tissue comprising:
an elongate shaft;
an expandable balloon coupled to the elongate shaft;
a light source to emit light through the balloon; and
viewing port of an endoscope, the viewing port configured to view tissue through the balloon.

2. The probe of claim 1, wherein the light source comprises a plurality of light sources.

3. The probe of claim 1, wherein the viewing port is located within the balloon.

4. The probe of claim 1, wherein the viewing port is located outside the balloon and configured to view the tissue through a first portion of the balloon and a second portion of the balloon.

5. The probe of claim 1, wherein the balloon comprises an optically transmissive material configured to allow the endoscope to image tissue through the balloon.

6. The probe of claim 1, wherein the balloon comprises a transparent material.

7. The probe of claim 1, wherein the balloon comprises a substantially transparent material configured to transmit light at red light, blue light, and green light and the endoscope is configured to generate a color image of the tissue through the balloon.

8. The probe of claim 1, wherein the balloon comprises sufficient transparency to view the tissue through the balloon with a resolving power of 100 μm through the endoscope.

9. The probe of claim 8, wherein the balloon material is sufficiently transparent to enable a visualization system to visualize blood flow, fluffy fibers, and anatomical structure to a resolving power of 100 μm.

10. The probe of claim 8, wherein the balloon comprises one or more of an elastomer, silicone, rubber, a thermoplastic rubber elastomer (e.g. ChronoPrene™), latex, polyethylene terephthalate (“PET”), urethane, polyurethane, polytetrafluoroethylene (“PTFE”), a conformal coating, a poly(p-xylylene) polymer, a chemically deposited poly(p-xylylene) polymer, Parylene™, nylon, poly(ether-b-amide), plasticizer-free poly(ether-b-amide), Pebax®, nylon elastomer.

11. The probe of claim 10, further comprising a coating on one or more of an inside or an outside of the balloon and optionally wherein the coating comprises one or more of starch, silicon, silica or corn starch.

12. The probe of claim 8, wherein the balloon comprises sufficient transparency to view the tissue through a first portion of the balloon and a second portion of the balloon with the resolving power of 100 μm, the first portion located near the tissue, the second portion located near the endoscope and closer to the endoscope than the first portion.

13. The probe of claim 1, wherein the balloon comprises a material with a layer configured to absorb at least about 50% of the light transmitted from the light source at a first wavelength to heat tissue with the balloon and to transmit at least about 50% of light from the tissue toward the endoscope at a second wavelength to image the tissue with the second wavelength and optionally wherein the probe is configured to inflate the balloon with a liquid to conduct heat from balloon.

14. The probe of claim 1, wherein the endoscope comprises a polychromatic light source to illuminate the tissue and generate a color image of the tissue.

15. The probe of claim 1, wherein the light source comprises an optical fiber extending toward a distal tip to emit light energy.

16. The probe of claim 1, wherein the light source comprises a laser diode located on the elongate shaft to emit light energy.

17. The probe of claim 1, wherein the balloon is configured to expand from a narrow profile configuration for insertion into a tissue space to an expanded profile to contact tissue.

18. The probe of claim 17, wherein the balloon in the narrow profile configuration comprises one or more of an approximately cylindrical shape within 25% of the probe diameter, a balloon comprising a diameter larger than a shaft of the probe and a tapered shape profile near a distal end of the balloon to facilitate advancement of the probe, or a balloon wrapped around the elongate shaft to decrease a cross-sectional size of the balloon.

19. The probe of claim 1, wherein the light source is configured to translate and rotate in relation to the balloon and the elongate shaft to coagulate tissue through the balloon.

20. The probe of claim 1, wherein the elongate shaft comprises a fluid flushing lumen extending to a flushing opening on a first side of the balloon and an evacuation opening extending to an evacuation lumen on the second side of the balloon, in order to establish fluid flow around the balloon to remove material between the tissue and the balloon.

21.-127. (canceled)
